SEQUENCE LISTING (18 sequences)

SEQ ID NO: 1 - DNA, 52 bp, Unknown
  Primer
aattctaatacgactcactatagggtgctatgtcacttccccttggttctct

SEQ ID NO: 2 - DNA, 29 bp, Unknown
  primer
agtggggggacatcaagcagccatgcaaa

SEQ ID NO: 3 - DNA, 20 bp, Unknown
  Capture probe
tggcctggtgcaataggccc

SEQ ID NO: 4 - DNA, 20 bp, Unknown
  Capture probe
cccattctgcagcttcctca

SEQ ID NO: 5 - DNA, 15 bp, Unknown
  primer
cgatcgagcaagcca

SEQ ID NO: 6 - DNA, 15 bp, Unknown
  Primer
cgagccgctcgctga

SEQ ID NO: 7 - DNA, 40 bp, Unknown
  primer
accgcatcgaatgcatgtctcgggtaaggcgtactcgacc

SEQ ID NO: 8 - DNA, 40 bp, unknown
  primer
cgattccgctccagacttctcgggtgtactgagatcccct

SEQ ID NO: 9 - DNA, 91 bp, Mycobacterium tuberculosis
tggacccgccaacaagaaggcgtactcgacctgaaagacgttatccaccatacggataggggatctcagtacacatcgatccggttcagcg

SEQ ID NO: 10 - DNA, 20 bp, Unknown
  primer; Nucleosides 9, 10, 11 and 12 are ribonucleoside bases
aaagatgtagagggtacaga

SEQ ID NO: 11 - DNA, 24 bp, Artificial sequence
  assay target sequence
aatctgtaccctctacatctttaa

SEQ ID NO: 12 - DNA, 28 bp, Unknown
  primer
ataatccacctatcccagtaggagaaat

SEQ ID NO: 13 - DNA, 28 bp, Unknown
  primer
tttggtccttgtcttatgtccagaatgc

SEQ ID NO: 14 - DNA, 49 bp, Escherichia coli
aattctaatacgactcactatagggagaggacggataaacggaactgga

SEQ ID NO: 15 - DNA, 18 bp, Escherichia coli
atgatgaaaacggcaacc

SEQ ID NO: 16 - DNA, 18 bp, Escherichia coli
ggtcggcttacggcggtg

SEQ ID NO: 17 - DNA, 23 bp, Escherichia coli
ctgtatgaacggtctggtctttg

SEQ ID NO: 18 - DNA, 50 bp, Artificial sequence
  assay test sequence
atcttagtcggaaatcgtattcaagtttatatgaccaggcagtagatact

(19) United States
(12) Patent Application Publication
(10) Pub. No.: US 2004/0110167 A1
(43) Pub. Date: Jun. 10, 2004

(54) Lateral flow system for nucleic acid detection

(76) Inventors: John C. Gerdes, Denver, CO (US); Roy R. Mondesire, Boulder, CO (US); Lara A. Hansen, Denver, CO (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/413,433

(22) Filed: Apr. 14, 2003

Related U.S. Application Data
(63) Continuation-in-part of application No. 09/705,043, filed on Nov. 2, 2000, now Pat. No. 6,649,378.
(63) Continuation of application No. 09/141,401, filed on Aug. 27, 1998, now Pat. No. 6,153,425.
Application No. 09/141,401 is a continuation-in-part of application No. 08/679,522, filed on Jul. 12, 1996, now Pat. No. 5,955,351.
(63) Continuation-in-part of application No. 09/061,757, filed on Apr. 16, 1998, now Pat. No. 6,291,166.
(60) Provisional application No. 60/000,885, filed on Jul. 13, 1995.
(60) Provisional application No. 60/041,999, filed on Apr. 16, 1997.

(51) Int. Cl. C12Q 1/70; C12Q 1/68; C12M 1/34; G01N 33/558

(52) U.S. Cl. 435/6; 435/5; 435/287.2; 436/514

(57) ABSTRACT

The invention provides a complete, one-step, fully functional, ready to use lateral flow assay device for the rapid, accurate detection of a target nucleic acid in a fluid sample, wherein the device contains all reagents necessary for the assay in an anhydrous format. The device comprises a sample receiving zone, a labeling zone, and a capture zone. The sample receiving zone may contain one or more oligonucleotides coupled to binding partners and reversibly bound to the capture zone membrane, the labeling zone comprises a visible moiety coupled to a ligand specific for one of the binding partners and reversibly bound to the labeling zone membrane, and the capture zone comprises an capture moiety specific for the second binding partner and immobilized on the capture zone membrane.

### RELATED APPLICATIONS

This application is a Continuation-in-Part of U.S. patent application Ser. No. 09/705,043, filed Nov. 2, 2000, is a Continuation of U.S. patent application Ser. No. 09/141,401, filed Aug. 27, 1998, which is a Continuation-in-Part of U.S. patent application Ser. No. 08/679,522, filed Jul. 12, 1996, now issued as U.S. Pat. No. 5,955,351. U.S. Patent application Ser. No. 08/679,552 claims priority to U.S. Provisional Application Serial No. 60/000,885, filed Jul. 13, 1995, now abandoned. This application is also a Continuation-in-Part of U.S. patent application Ser. No. 09/061,757, filed Apr. 16, 1998, which claims priority to U.S. Provisional Application Serial No. 60/041,999, filed Apr. 16, 1997. All of the above-referenced applications are specifically incorporated herein by reference.

### BACKGROUND OF THE INVENTION

1. Field of the Invention

This invention relates to the general fields of molecular biology and medical science, and specifically to a lateral flow device for rapid and accurate detection of target nucleic acid sequences, wherein the device contains all required reagents for the assay.

2. Description of the State of the Art

The use of nucleic acid probe tests based on hybridization in routine clinical laboratory procedures is hindered by lack of sensitivity. The ability to amplify nucleic acids from clinical samples has greatly advanced nucleic acid probe technology, providing the sensitivity lacking in earlier versions of non-isotopic assays. Sensitivity afforded by oligonucleotide probe tests utilizing nucleic acid amplification now exceeds that of any other method; Nucleic acid amplification procedures can detect a single copy of a specific nucleic acid sequence. Routine detection and identification of specific gene sequences has extremely broad applications in a number of settings and industries.

The major barrier for the transfer of technology to routine field testing is the absence of an economical and easy-to-use system or apparatus. In order to compete in today's cost conscious environment, genetic based testing must provide for high throughput while incorporating adequate controls and safeguards to prevent false positive results due to sample cross-contamination.

Current technology involves several steps, although recent developments are directed toward automating systems for detection of the amplified target sequence. The first step, extraction of nucleic acids, is accomplished in a variety of ways, for example, phenol extraction, chaotropic reagent extraction, chromatographic purification such as purification on silica membranes (WO 95/01359, specifically incorporated herein) and ultracentrifugation (Maniatis, et al., Molecular Cloning: A Laboratory Manual, Cold Spring Harbor Laboratory, Cold Spring Harbor, N.Y. (1982) specifically incorporated herein by reference). Phenol is a well-established health hazard and requires special handling for waste removal. The extraction method is also tedious and labor intensive. Ultracentrifugation often requires the use of expensive and hazardous chemicals as well as the use of sophisticated and costly equipment. The process often requires long run times, sometimes involving one or more days of centrifugation. The easiest and fastest method is separation using chromatography purification.

The second step, the amplification of the target nucleic acid, employs a variety of enzymes known as polymerases and ligases. Polymerase chain reaction (PCR) is the most commonly used amplification technique. The general principles and conditions for amplification of nucleic acids using PCR are quite well known in the art, the details of which are provided in numerous references including U.S. Pat. No. 4,683,195, U.S. Pat. No. 4,683,202 and U.S. Pat. No. 4,965,188, all to Mullis, et al., and all of which are specifically incorporated herein by reference. Thus, the details of PCR technology are not included herein. Other approaches include ligase chain reaction, Qβ replicase, strand displacement amplification (SDA), transcription mediated iso CR cycling probe technology, nucleic acid sequence-based amplification (NASBA) and cascade rolling circle amplification (CRCA).

A current protein detection technology for antigen-antibody assays involves the use of microparticles. Furthermore, a variety of microparticle strategies for dipstick detection in antigen-antibody assays are currently available, for example, a currently marketed at-home pregnancy test (U.S. Pat. No. 5,141,850 to Cole et al., specifically incorporated herein by reference). Such tests use dyed particles that form a visible line following a specific antigen-antibody reaction.

The third and final step, detection of amplified nucleic acid for clinical use relies largely on hybridization of the amplified product and detection with a probe labeled with a variety of enzymes and luminescent reagents. U.S. Pat. No. 5,374,524 to Miller, which is specifically incorporated herein by reference, describes a nucleic acid probe assay that combines nucleic acid amplification and solution hybridization using capture and reporter probes. These techniques require multiple reagents, several washing steps, and specialized equipment for detection of the target nucleic acid. Moreover, these techniques are labor intensive and require technicians with expertise in molecular biology.

The use of probes comprised of oligonucleotide sequences bound to microparticles is well known and illustrated in the prior art. The mechanism for attachment of oligonucleotides to microparticles in hybridization assays and for the purification of nucleic acids is also well known. European Patent No. 200133, which is specifically incorporated herein, describes the attachment of oligonucleotides to water-insoluble particles less than 50 micrometers in diameter used in hybridization assays for the capture of target nucleotides. U.S. Pat. No. 5,387,512 to Wu, which is specifically incorporated herein by reference, describes the use of oligonucleotide sequences covalently bound to microparticles as probes for capturing PCR amplified nucleic acids. U.S. Pat. No. 5,328,825 to Findlay, which is specifically incorporated herein by reference, also describes an oligonucleotide linked by way of a protein or carbohydrate to a water-insoluble particle. The oligonucleotide probe is covalently coupled to the microparticle or other solid support. The sensitivity and specificity of all of the above-reference patents is based on hybridization of the oligonucleotide probe to the target nucleic acid.

The use of incorporated non-radioactive labels into amplification reactions for the detection of nucleic acids is also well known in the art. Nucleic acids modified with biotin (U.S. Pat. No. 4,687,732 to Ward et al.; European Patent No. 063879; both of which are specifically incorporated herein by reference), digoxigenin (European Patent No. 173251, which is specifically incorporated herein) and other haptens have also been used. For example, U.S. Pat. No. 5,344,757 to Graf, which is specifically incorporated herein by reference, uses a nucleic acid probe containing at least one hapten as a label for hybridization with a complementary target nucleic acid bound to a solid membrane. The sensitivity and specificity of these assays is based on the incorporation of a single label in the amplification reaction which can be detected using an antibody specific to the label. The usual case involves an antibody conjugated to an enzyme. Furthermore, the addition of substrate generates a calorimetric or fluorescent change that can be detected with an instrument.

Several point-of-care approaches have been developed for detection of molecules of interest. Two of these methods are the immunochromatographic (lateral flow) and flow-through devices. In lateral flow assays the sample flows laterally through a microporous membrane from the zone of application to a region on the membrane where a specific capture reagent is present. The analyte of interest is generally visualized by direct visualization of visible entities at the capture reagent line. Lateral flow assays have been used to detect a variety of analytes, including antigens from various microorganisms, antibodies, tumor markers, cardiac markers, and drugs of abuse. However, there are very few disclosures for the detection of nucleic acids using lateral flow assays. See, for example, U.S. Pat. No. 5,869,252, U.S. Pat. No. 6,037,127, and U.S. Published Patent Application No. 2001/0036634 A1, each of which is specifically incorporated herein by reference.

Still, the above-described approaches are labor intensive and involve many steps and washes. In addition, the above-described approached require special and costly equipment for the detection of the target nucleic acid, require trained staff, and take several hours to complete. Several patents have issued which deal with automation of the processes of amplification and subsequent detection of the amplicon. These patents use specialized equipment and are still based on the principle of hybridization and immunoassay technology. For example, European Patent No. 320308, which is specifically incorporated herein by reference, describes a system detecting target nucleic acids amplified by the ligase chain reaction.

Nucleic acid probe technology has developed rapidly in recent years as the scientific community has discovered its value for detection of various diseases, organisms or genetic abnormalities. Amplification techniques have provided the sensitivity to qualitatively determine the presence of even minute quantities of nucleic acid. The drawback to wide spread use of this technology is the possibility of cross contamination of samples since the test is so sensitive. The cost of nucleic acid based testing is high, as it requires highly skilled technicians and sophisticated equipment. Automated approaches eliminate the need for specially trained personnel, however, the cost of the equipment is very high and the possibility of contamination still exists since many samples will be processed by the same equipment.

There is still a need, therefore, for methods and devices which provide for the rapid and accurate detection of amplified and nonamplified nucleic acid sequences while further being simple, economical and ready to use. There also remains a need for a device that also significantly decreases the possibility of cross-contamination of samples.

### SUMMARY OF THE INVENTION

One aspect of this invention provides a complete, one-step, fully functional, ready to use lateral flow assay device for the rapid, accurate detection of one or more target nucleic acids in a fluid sample, wherein the device contains all reagents necessary for the assay in an anhydrous format. More specifically, one embodiment of this invention provides a lateral flow assay device for detecting the presence or absence of a single-stranded target nucleic acid in a fluid sample, said device comprising a test strip having a first and second end and comprising:

a sample receiving zone at or near said first end for receiving an aliquot of said sample and comprising a porous material having first and second oligonucleotide probes coupled to first and second binding partners, respectively, wherein said probes specifically hybridize to said target nucleic acid to form a complex having said first and second binding partners, said sample receiving zone being in lateral flow contact with

a labeling zone comprising a porous material having at least a first visible moiety reversibly bound thereto and coupled to a first ligand which specifically binds to said first binding partner to form a visible complex, said labeling zone being in lateral flow contact with

a capture zone comprising a microporous membrane which contains in a portion thereof a first capture moiety immobilized thereto which specifically binds said second binding partner, said capture zone being in lateral flow contact with

an absorbent zone positioned at or near the second end of said test strip, wherein said visible complex is captured by said capture moiety in said portion of the capture zone.

In one embodiment, the visible moiety comprises a ligand coupled to a colored microparticle.

When the device is designed for the detection of two or more target nucleic acids, the sample receiving zone comprises a first and second oligonucleotide probe specific for each target nucleic acid, the labeling zone comprises a distinguishable first visible moiety specific for each target nucleic acid, and the capture zone comprises a specific capture moiety for capturing each target nucleic acid. The capture moieties for the different target nucleic acids are immobilized in distinct regions of the capture zone.

An alternate embodiment of a lateral flow assay device of this invention provides a lateral flow assay device comprising a test strip for detection of the presence or absence of one or more target nucleic acids in a fluid sample, wherein the target nucleic acid is coupled to a first binding partner. In this embodiment, the test strip comprises a sample receiving zone for receiving an aliquot of said sample and comprising a porous material having an oligonucleotide probe coupled to a second binding partner, wherein said probe is reversibly bound to said porous material and specifically hybridizes to said target nucleic acid to form a complex comprising said first and second binding partners.

Yet another embodiment of a lateral flow assay device of this invention provides a lateral flow assay device comprising a test strip for detection of the presence or absence of one or more target nucleic acids in a fluid sample, wherein said target nucleic acid is coupled to a first and second binding partner. In this embodiment, the test strip comprises a sample receiving zone for receiving an aliquot of said sample and comprising a porous material.

The sample receiving zone, the labeling zone, and the absorbent pad can each be affixed to the capture zone membrane. Alternatively, the sample receiving zone, the labeling zone, the capture zone and the absorbent pad can be contiguous separate materials provided that the sequential zones are in lateral flow contact with each other.

Any of the test strips of the devices described herein may be completely sheathed or sealed in a transparent film, except for a portion of the sample receiving zone, to prevent contamination during the assay. Such sealing does not compromise the integrity of the device.

In another embodiment, the device is affixed to a heating sheet so that the device can be heated during use.

Another aspect of this invention provides a method for detecting the presence or absence of one or more target nucleic acids in a fluid sample. More specifically, one embodiment of this method comprises:

(a) applying said sample to a sample receiving zone of a lateral flow test strip of a lateral flow assay device, wherein prior to said application said nucleic acid present in a double-stranded form are rendered single-stranded, wherein said sample wicks sequentially from said sample receiving zone to a labeling zone and to a capture zone of said test strip, wherein said sample receiving zone comprises first and second oligonucleotide probes coupled to first and second binding partners, respectively, and reversibly bound to said test strip, wherein said probes are released from said test strip and specifically hybridize to said target nucleic acid upon contact with said sample to form a complex comprising said first and second binding partners, said labeling zone comprises at least a first visible moiety coupled to a first ligand and reversibly bound to said test strip, wherein said first ligand specifically binds said first binding partner, and said capture zone comprises a capture moiety immobilized to a portion of said test strip, wherein said capture moiety specifically binds said second binding partner; and

(b) detecting the presence of said first visible moiety in said portion of said capture zone.

The assays and devices of the invention are applicable for the detection of extracted non-amplified target nucleic acids as well as amplified target nucleic acids, and can be performed under high or low stringency conditions. The assays are also suitable for determining Watson-Crick complementarity.

The lateral flow assay devices of this invention enable rapid turnaround time in the detection of target nucleic acids, in that the results of the assay are obtained within 10 to 300 seconds from commencement of the assay.

An alternate embodiment of an assay of this invention provides a method for detecting the presence or absence of a target nucleic acid in a fluid sample, wherein the target nucleic acid is coupled to a first binding partner to provide a labeled target nucleic acid. In this embodiment, the labeled target nucleic acid is applied to a sample receiving zone of a lateral flow test strip comprising an oligonucleotide probe coupled to a second binding partner and reversibly bound to said test strip.

Yet another embodiment of an assay of this invention provides a method for detecting the presence or absence of a target nucleic acid in a fluid sample, wherein the target nucleic acid is coupled to a first and second binding partner to provide a labeled target nucleic acid. In this embodiment, the sample receiving zone does not contain oligonucleotide probes specific for the target nucleic acid, and the sample wicks to the labeling zone and capture zone as described above.

In one embodiment, the device is affixed to a heating sheet, and the assay further comprises heating the device to a temperature between about 25 and 90° C. during the assay.

This invention further provides novel, self-contained devices that integrate nucleic acid extraction, specific target amplification and detection methodologies into a single device, permitting rapid and accurate nucleic acid sequence detection. The present invention is applicable to all nucleic acids and derivatives thereof. According to one embodiment, the method of detecting nucleic acids takes place in a self-contained device of this invention.

Additional advantages and features of this invention shall be set forth in part in the description that follows, and in part will become apparent to those skilled in the art upon examination of the following specification or may be learned by the practice of the invention. The features and advantages of the invention may be realized and attained by means of the instrumentalities, combinations, and methods particularly pointed out in the appended claims.

### BRIEF DESCRIPTION OF THE FIGURES

The file of this patent contains at least one drawing executed in color. Copies of this patent with color drawings will be provided by the Patent and Trademark Office upon request and payment of the necessary fee.

The accompanying drawings, which are incorporated in and form a part of the specification, illustrate non-limiting embodiments of the present invention, and together with the description serve to explain the principles of the invention.

In the Figures:

FIG. 1 illustrates one embodiment of a test strip used in a lateral flow device, wherein the sample receiving zone material, the labeling zone material, and the absorbent pad are each affixed to the capture zone membrane, which in turn is affixed to a semi-rigid or rigid support.

FIG. 2 illustrates another embodiment of a test strip used in a lateral flow device, wherein the sample receiving zone material, the labeling zone material, the capture zone, and the absorbent pad are each affixed to a semi-rigid or rigid support.

FIGS. 3A and 3B illustrate a lateral flow assay wherein the test strip of the lateral flow device wherein the sample receiving zone comprises two oligonucleotide probes and receives a non-labeled target nucleic acid.

FIGS. 4A and 4B illustrate a lateral flow assay wherein the test strip of the lateral flow device wherein the sample receiving zone comprises one oligonucleotide probe and receives a singly-labeled target nucleic acid.

FIGS. 5A and 5B illustrate a lateral flow assay wherein the test strip of the lateral flow device wherein the sample receiving zone does not contain any oligonucleotide probes and receives a doubly-labeled target nucleic acid.

FIG. 6 is a perspective view of a self-contained device integrating nucleic acid extraction, amplification and detection, illustrating each of the three device rotational positions: closed (A); open (B); and elute (C).

FIG. 7 is a schematic of the preferred sealing mechanism, illustrating each of the three device rotational positions: closed (A); open (B); and elute (C), which are enlargements of the encircled portions of positions (A), (B), and (C) as shown in FIG. 6.

FIG. 8 is a top plan view of the device shown in FIG. 6, position A along line 3-3, showing the hinged cover in the open position.

FIG. 9 is a side cross-sectional view of the hinged cover in the closed position and the reaction bead contained within a reaction bead chamber having an integral knife-edge.

FIG. 10 is a top cross-sectional view of the aperture section of the second hollow elongated cylinder.

FIG. 11 depicts the relative position of the absorbent pad and membrane strip having microparticles and capture zones.

FIG. 12 depicts a sequential operating sequence of one embodiment of a self-contained device.

FIG. 13 is side cross-sectional view of the alternate embodiment of the instant invention comprising a matrix tube inserted inside of the PCR tube with the cap in the opened position.

FIG. 14 is a side cross-sectional view of the matrix tube of an alternate embodiment of the invention having a solid phase matrix sandwiched between an upper and lower screen.

FIG. 15 depicts a side cross-sectional view of the PCR tube of an alternate embodiment of the invention, said tube having a specially designed lid.

FIG. 16 depicts a side cross-sectional view of the reagent cell of an alternate embodiment of the invention having a plurality of pouches.

FIG. 17 is a side view of the result stick of an alternate embodiment of the invention.

FIG. 18 is a side cross-sectional view of the alternate embodiment of the instant invention comprising a matrix tube inserted within the PCR tube and the cap in the closed position, and a top plan view of the lid of the alternate embodiment of the invention.

FIG. 19 is a side cross-sectional view of the alternate embodiment of the invention, showing detection via a result stick.

FIG. 20 depicts a nucleic acid sequenced-based amplification (NASBA) strategy.

FIG. 21 illustrates the reagents and their respective interactions in the amplification chamber of the device in a strand displacement amplification (SDA) strategy.

FIG. 22 depicts reagents and their respective interactions in an alternative strand displacement amplification (SDA).

FIG. 24 depicts the reagents and their respective interactions in a cycling probe assay.

FIG. 23 illustrates the detection results of isothermal amplification and detection with bifunctionally labeled amplified target sequence using strand displacement amplification.

FIG. 25 shows the detection results of a lateral flow assay using cycling probe technology.

FIG. 26 shows the detection results of an alternate lateral flow assay.

FIG. 27 shows the results of detection by amplification with a single labeled primer followed by hybridization with a probe containing a single label.

FIG. 28 shows the results of CRCA methodology use for the detection of nucleic acid target sequences in terms of lateral flow detection strips versus gel electrophoresis.

FIG. 29 shows lateral flow test strips after an assay, wherein strip 1 is a positive control (no laminate coating), and strips 2-6 are laminated with a clear polyester with acrylic adhesive.

FIG. 30 shows lateral flow test strips obtained following assays for the detection of S. tryphimurium. The strips are shown in increasing levels of detection probe mix concentration.

FIG. 31 shows the effects of heat on the integrity of lateral flow devices of the invention. A positive strip and negative strip is shown at each temperature tested.

### DETAILED DESCRIPTION OF THE INVENTION

This invention provides rapid and accurate methods for assessing the presence or absence of one or mote target nucleic acids in a sample, and devices for conducting such methods. Accordingly, one aspect of this invention provides a one step, ready to use, fully functional lateral flow assay device comprising a lateral flow test strip for the rapid, accurate detection of one or more target nucleic acids in a fluid sample, wherein the device contains all required reagents for the assay in an anhydrous format. Results from the methods and devices disclosed herein can be read with the naked eye directly from the assay device without having to contact the test strip with a chemical or a visualizing agent in order to detect the results.

By “lateral flow” it is meant that a sample suspected of containing a target nucleic acid is placed on a test strip comprising a chromatographic material and the sample is wicked laterally through of the test strip by capillary action and binds to various reagents in the strip.

Accordingly, one embodiment of this invention provides a lateral flow assay device for detecting the presence or absence of a single-stranded target nucleic acid in a fluid sample, said device comprising a test strip having a first and second end and comprising:

a sample receiving zone at or near said first end for receiving an aliquot of said sample and comprising a porous material having first and second oligonucleotide probes coupled to first and second binding partners, respectively, wherein said probes specifically hybridize to said target nucleic acid to form a complex having said first and second binding partners, said sample receiving zone being in lateral flow contact with

a labeling zone comprising a porous material having at least a first visible moiety reversibly bound thereto and coupled to a first ligand which specifically binds to said first binding partner to form a visible complex, said labeling zone being in lateral flow contact with

a capture zone comprising a microporous membrane which contains in a portion thereof a first capture moiety immobilized thereto which specifically binds said second binding partner, said capture zone being in lateral flow contact with

an absorbent zone positioned at or near the second end of said test strip, wherein said visible complex is captured by said capture moiety in said portion of the capture zone.

As used herein, the term “target nucleic acids” refers to the nucleic acid molecule that may be amplified or non-amplified for detection with the presented methods. The “target” molecule can be purified, partially purified, or present in an unpurified state in the sample.

The term “test strip” refers to a chromatographic-like medium upon which an assay of this invention is preformed. Briefly, the test strip contains in sequential order a “sample receiving zone” at the proximal end for the application of the test sample, a “labeling zone” comprising visible moieties which are visible to the naked eye, a “capture zone” which contains an immobilized capture moiety that captures and retains the target nucleic acid, and an absorbent pad at the distal end to helps draw the sample through the test strip. The visible moieties provide means for detecting the presence of the target nucleic acid in the capture zone. The visible moieties are coupled to a ligand that specifically binds a binding partner coupled to or complexed with the target nucleic acid. These visible moieties bind the target nucleic acid as the fluid sample passes through the labeling zone and are carried to the capture zone by the liquid flow. When the target nucleic acid/visible moiety complex reaches the capture zone, a capture moiety, which is specific for a second binding partner coupled to or complexed with the target nucleic acid, captures and retains the complex.

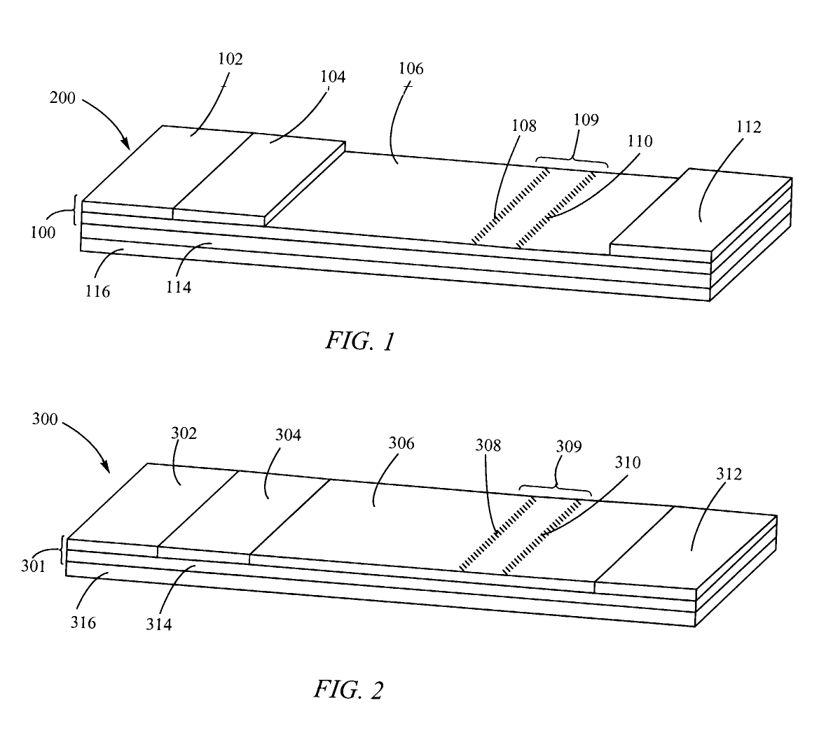

One embodiment of a test strip 100 of a lateral flow device 200 of this invention is shown in FIG. 1. In the embodiment shown in FIG. 1, the capture zone membrane 106 extends the length of the test strip, and the sample receiving zone material 102 is affixed to the capture zone membrane 106. The sample receiving zone 102 serves to receive a fluid sample which may contain the target nucleic acid and to begin the flow of the sample along the test strip. The sample receiving zone 102 is prepared from a natural or synthetic porous or macroporous material which is capable of conducting lateral flow of the fluid sample. A porous or macroporous material suitable for purposes of this invention generally has a pore size greater than 12 μm. Examples of porous materials include, but are not limited to, glass, cotton, cellulose, polyester, rayon, nylon, polyethersulfone, and polyethylene.

The sample zone receiving material must be a material that does not irreversibly bind nucleic acids (i.e., the oligonucleotide probes and the target nucleic acid). Rather the sample receiving zone material must sufficiently retain the oligonucleotide probe on or within the sample receiving zone in an anhydrous form prior to use of the lateral flow device, but must also release the oligonucleotide probe upon contact with the fluid sample and also allow lateral flow of the target nucleic acid. The solution used to prepare the fluid sample also plays a role in rehydrating and thus releasing the oligonucleotide probes from the sample zone receiving material, as discussed below.

In one embodiment, the sample receiving zone material 102 contains anhydrous forms of one or more oligonucleotide probes, each coupled to a different binding partner, for hybridizing to the target nucleic acid. Alternatively, the sample receiving zone serves simply to receive a test sample containing a target nucleic acid coupled to two different binding partners and to begin the flow of the sample along the test strip.

The term “oligonucleotide probe” refers to a nucleic acid which has a sequence complementary to a portion of the target nucleic acid and which is further coupled to a binding partner. The oligonucleotide probe may either be reversibly bound to the sample receiving zone of a test strip, and/or may be used to label the target nucleic acid prior to introduction to the lateral flow system as described herein (in the latter case the oligonucleotide probe is also referred to as a “primer”).

The terms “complementary” or “complementarity” are used in reference to nucleic acids (i.e., a sequence of nucleotides) related by the well-known base-pairing rules that A pairs with T and C pairs with G. For example, the sequence 5′-A-G-T-3′, is complementary to the sequence 3′-T-C-A-5′. Complementarity can be “partial,” in which only some of the nucleic acid bases are matched according to the base pairing rules. On the other hand, there may be “complete” or “total” complementarity between the nucleic acid strands when all of the bases are matched according to base pairing rules. The degree of complementarity between nucleic acid strands has significant effects on the efficiency and strength of hybridization between nucleic acid strands as known well in the art. This is of particular importance in detection methods that depend upon binding between nucleic acids, such as those of the invention. The term “substantially complementary” refers to any probe that can hybridize to either or both strands of the target nucleic acid sequence under conditions of high stringency as described below or, preferably, in polymerase reaction buffer, heated to about 956° C. and then cooled to about room temperature (e.g., 250° C.±3° C.).

The probes may be reversibly bound to the sample receiving zone material directly by vacuum transfer, or by other well known methods such as drying and desiccation. In this embodiment, the oligonucleotide probe functions to label the target nucleic acid with a binding partner by hybridizing with it as it passes through the sample receiving zone of the test strip.

As used herein, the term “binding partner” refers to a member of a pair of molecules and/or compositions capable of recognizing a specific structural aspect of another molecule or composition, wherein the binding partners interact with each other by means of a specific, noncovalent or covalent interaction. Examples of such binding partners and corresponding molecules or compositions include, but are not limited to, any of the class of immune-type binding pairs, such as antigen/antibody or hapten/anti-hapten systems; and also any of the class of nonimmune-type binding pairs, such as biotin/avidin, biotin/streptavidin, digoxigenin/anti-digoxigenin F(ab′)₂, folic acid/folate binding protein, complementary nucleic acid segments, protein A or G/immunoglobulins, lectin/carbohydrate, substrate/enzyme, inhibitor/enzyme, virus/cellular receptor; and binding pairs which form covalent bonds, such as sulfhydryl reactive groups including maleimides and haloacetyl derivatives, and amine reactive groups such as isotriocyanates, succinimidyl esters and sulfonyl halides. Other binding partners include steroids, halogens and 2,4-dinitrophenyl.

The labeling zone 104 comprises a material that is capable of conducting lateral flow and is in lateral flow contact with the sample receiving zone 102. In the embodiment shown in FIG. 1, the labeling zone material 104 is affixed to the capture zone membrane 106 on the same side as the sample receiving zone. Materials suitable for the labeling zone material include, but are not limited to, porous or macroporous materials such as glass (e.g., borosilicate glass fiber), cotton, cellulose, polyester, polyethylene, rayon or nylon. The labeling zone comprises at least a first (“test”) visible moiety (e.g., a colored microparticle) which is reversibly bound to the matrix and is coupled to a first ligand. In the present invention, the ligands are specific for discrete binding partners coupled to or complexed with amplified or non-amplified target nucleic acids. The labeling zone material 104 must sufficiently retain the visible moieties in an anhydrous form prior to use of the lateral flow device, but must also release the visible moieties upon contact with the fluid sample and allow lateral flow of the target nucleic acid both before and after it becomes bound to the visible moiety.

The labeling zone material 104 may also comprise a second visible moiety (i.e., a “control” visible moiety) which is reversibly bound to the labeling zone material. The control moiety is carried through to the capture zone along with the liquid flow. The control visible moiety does not contain a ligand specific for the target nucleic acid binding partner. Rather, the control visible moiety is coupled to a control ligand which binds its specific binding partner that is immobilized in a separate “control” portion of the capture zone. The control visible moiety is useful for verifying that the flow of fluid sample is as expected and that the microparticles have been successfully released from the labeling zone. The control visible moieties may be the same or a different color than the test visible moieties. If different colors are used, ease of reading the results is enhanced.

The capture zone membrane 106 comprises a microporous material which is capable of conducting lateral flow and is in lateral flow contact with the labeling zone material. Materials suitable for the capture zone membrane include, but are not limited to, microporous materials having a pore size from about 0.05 μm to 12 μm, such as nitrocellulose, polyethersulfone, polyvinylidine fluoride, nylon, charge-modified nylon, and polytetrafluoroethylene. The capture zone 109 comprises a test capture region 108 comprising a first (“test”) capture moiety that specifically binds a second binding partner coupled to or complexed with the target nucleic acid. That is, the test capture moiety and the second binding partner a members of a binding pair that specifically recognize each other. The arrangement of the first capture moiety in the capture zone may be, for example, in the form of a dot, line, curve, band, cross, or combinations thereof.

The capture zone 109 may also contain a second (“control”) capture moiety is a region 110 which specifically binds the ligand coupled to the control visible moiety. The arrangement of the second capture moiety in region 110 may be in the form of a dot, line, curve, band, cross, or combinations thereof. In one embodiment, as shown in FIG. 1, the immobilized second capture moieties are in a region 110 that is separate from the region 108 that contains immobilized first capture moieties. Alternatively, the first and second capture moieties are contained within the same region. In this embodiment, the first and second visible moieties contain microparticles of different colors (e.g., blue and yellow), and the detection of a third color (e.g., green) in the capture zone indicates a positive result (i.e., the presence of the target nucleic acid). The control region 110 is helpful in that appearance of a color in the control region 110 signals the time at which the test result can be read, even for a negative result. Thus, when the expected color appears in the control region 110, the presence or absence of a color in the test region 108 can be noted.

Methods of immobilizing the capture moieties to the membrane are well known in the art. In general, the test and control capture moieties can be dispensed onto the membrane as spaced parallel lines (i.e., to form regions 108 and 110, respectively) with a linear reagent dispensing system using a solution of the test capture moiety diluted with a suitable buffer and a solution of the control capture moiety diluted with a suitable buffer. After air drying for a suitable period of time, the membrane is blocked with an appropriate buffer and stored in a desiccator until assembly of the test strip.

The absorbent pad or zone 112 is an absorbent material that is placed in lateral flow contact with the capture zone at the distal end of the test strip. In the embodiment shown in FIG. 1, the absorbent pad 112 is affixed to the capture zone membrane 106 on the same side of the membrane as the sample receiving zone and the labeling zone. The absorbent pad 112 helps to draw a test sample from the sample receiving zone to the distal end of the test strip by capillary action. Examples of materials suitable for use as an absorbent pad include any absorbent material, include, but are not limited to, nitrocellulose, cellulose esters, glass (e.g., borosilicate glass fiber), polyethersulfone, and cotton.

In the embodiment illustrated in FIG. 1, the capture zone membrane 106 is affixed to a rigid or semi-rigid support 114, which provides structural support to the test strip. The support can be made of any suitable rigid or semi-rigid material, such as poly(vinyl chloride), polypropylene, polyester, and polystyrene. The membrane 106 may be affixed to the support 114 by any suitable adhesive means such as with a double-sided adhesive tape. Alternatively, the support 114 may be a pressure sensitive adhesive laminate, e.g., a polyester support having an acrylic pressure sensitive adhesive on one side that is optionally covered with a release liner prior to application to the membrane.

Support 114 may optionally be affixed to a heating sheet 116, as shown in FIG. 1. The heating sheet may be any material suitable for conducting heat to the test strip, such as copper, aluminum, or titanium. The heating sheet 116 allows the lateral flow assays to be conducted at temperatures above room temperature, for example to increase the stringency of the assay or to determine Watson-Crick complementarity.

An alternative embodiment of a lateral flow device 300 of this invention is shown inn FIG. 2. In this embodiment, the sample receiving zone material 302, the labeling zone material 304, the capture zone membrane 306, and the absorbent pad 316 are each affixed to a rigid or semi-rigid support 314. As shown, sample receiving zone material 302 overlaps with labeling zone material 304 to allow for lateral flow contact therebetween. Similarly, the labeling zone material 304 overlaps with the capture zone membrane 306, and the capture zone membrane 306 overlaps with the absorbent pad 312. While it is not required that materials 302, 304, 306, and 312 overlap as describe, these materials must at least be in physical contact in the sequence shown in FIG. 2 such that the test sample can wick along the test strip 301 without interruption. Again, the support 314 may optionally be affixed to a heating sheet 316.

In another embodiment of this invention, test strip of the lateral flow devices of this invention are sheathed in a transparent film, provided that a portion of the sample receiving zone is left uncovered to allow application of the fluid sample to the test strip. For example, the test strip may be wrapped or sheathed using a clear polyester film having a pressure-sensitive adhesive coated on one side of the film by pressing the adhesive side of the film to all surfaces of the device except for a predetermined portion of the sample receiving zone. Other materials that could be used to wrap the device include any clear polymer that can withstand elevated temperatures (e.g., 95° C. or greater for at least 3-5 minutes) such as the temperatures used when the assay is performed in conjunction with the heating sheet. Thus, other examples of suitable wrapping materials include polycarbonates (e.g., Lexan), heat resistant acrylics (e.g., polymethylmethacrylate), butyrates (e.g., cellulose acetate butyrate), polystyrene, polypropylene, and glycol modified polyethylene terphthalate. If the lateral flow device comprises a support 114, the portion of the device wrapped in the film includes both the test strip and the support 114. Wrapping the device with a clear film helps to prevent contamination of the sample during an assay while still allowing visual monitoring of the capture zone.

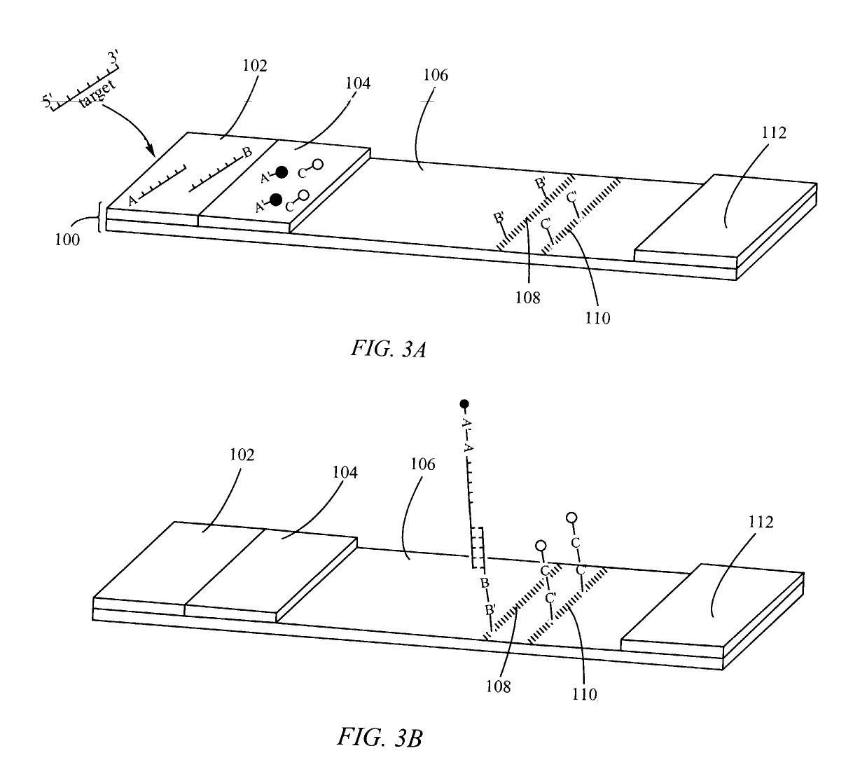

This invention also provides a method for detecting the presence or absence of one or more target nucleic acids in a fluid sample. One embodiment of an assay of this invention is illustrated in FIGS. 3A and 3B. The method illustrated in FIGS. 3A and 3B illustrates an embodiment wherein an unlabeled target nucleic acid is detected using a lateral flow device comprising two oligonucleotide probes reversibly bound to a membrane. Beginning with FIG. 3A, the assay device comprises test strip 100 having sample receiving zone 102. In this embodiment, sample receiving zone 102 comprises a first oligonucleotide probe coupled to a first binding partner (A) and a second oligonucleotide probe coupled to a second binding partner (B). Prior to applying the fluid sample, which may contain the target nucleic acid, to the sample receiving zone 102, any nucleic acid present in the sample in a double stranded form is rendered single stranded by any denaturing method known in the art. The fluid sample is subsequently to the sample receiving zone. Alternatively, the target nucleic acid can be amplified prior to application to the sample receiving zone using any nucleic acid amplification method, such as those described herein. Test strip 100 also contains a first visible moiety reversibly bound to the labeling zone material 104 and coupled to ligand (A′). Ligand (A′) is designed to specifically recognize and bind to binding partner (A) coupled to the first oligonucleotide probe. Test strip 100 further comprises capture zone 108 containing capture moieties (B′) immobilized on the capture zone membrane. Capture moiety (B′) is designed to specifically recognize and bind to binding partner B coupled to the second oligonucleotide probe.

The solution used to prepare the fluid sample contains reagents that rehydrate the oligonucleotide probes, thereby releasing the probes from the test strip. For example, the probes can be released form the material simply by rehydrating with water. It is known in the art that additional “release agents” such as surfactants, gelatin (e.g., fish skin gelatin), polymers (e.g., polyvinyl pyrrolidone), Tween 20, and sugars (e.g., sucrose or sorbitol) can facilitate the release of the probes. Thus, when the fluid sample is applied to the sample receiving zone, the target nucleic acid in the sample specifically hybridizes with the first and second oligonucleotide probes to form a complex comprising first and second binding partners (A) and (B). The target nucleic acid/visible moiety complex continues flowing with the fluid sample along the test strip 100 by capillary action in the direction of the labeling zone 104.

As the fluid sample moves through the labeling zone 104, the visible moiety coupled to ligand (A′) is released form the labeling zone material and ligand (A′) and binds to binding partner (A) of the complexed nucleic acid/visible moiety complex. The bound visible moiety thus flows along with the complex in the direction of the capture zone 108 as shown in FIG. 3B. Upon reaching the capture zone 108, binding partner (B) of the complexed nucleic acid is captured and immobilized in capture zone 108 by capture moiety (B′). Thus, if the target nucleic acid is present in the sample, the first visible moieties will be collected and bound in the capture zone 108, forming a visible signal such as a colored line, which can be detected with the naked eye without having to contact the test strip with a visualizing reagent or chemical. Continued movement of the sample fluid draws excess reagents and unbound material (e.g., unbound test visible moieties) past the capture zone to the absorbent pad 112.

The assay outlined in FIGS. 3A and 3B can also incorporate the use of a control visible moiety to verify that the microparticles were successfully released from the test strip. Thus, with reference to FIG. 3A, labeling zone 104 can further comprise a second (control) visible moiety reversibly bound to the labeling zone membrane and coupled to ligand (C), and capture zone 110 can comprise capture moiety (C′) immobilized to the capture zone membrane in region 110. Ligand (C) and capture moiety (C′) are members of a binding pair that specifically recognize and bind to each other. During the assay illustrated in FIGS. 3A and 3B, the control visible moiety flows along with the fluid sample in the direction of the capture zone 110. Upon reaching the capture zone 110, binding partner (C) of the control visible moiety collect and are captured in capture zone 110 by capture moiety (C′), thus forming a visible, detectable signal, e.g., a colored line. The control visible moieties may be the same or a different color than the test visible moieties. If different colors are used, ease of reading the results is enhanced. In an alternative embodiment, capture zones 108 and 110 overlap. In this embodiment the first and second (control) visible moieties contain visible moieties (e.g., microparticles) of different colors (e.g., blue and yellow), and the detection of a third color (in this case, green) in the capture zone indicates the presence of the target nucleic acid.

In the assays of this invention, it is important that the concentration of the second oligonucleotide probe (coupled to binding partner (B)) in the sample receiving zone 102 is in an amount sufficient to hybridize with the target nucleic acid and produce a visible signal in the capture zone, but is not so high that the second probe competes with the complex for binding to the first capture reagent (B′) in the capture zone 108.

In another embodiment, the assay described with reference to FIGS. 3A and 3B can be used to detect two or more target nucleic acids. In this embodiment, the sample receiving zone 102 comprises a first and second oligonucleotide probe specific for each target nucleic acid, the labeling zone 104 comprises a specific and distinguishable first visible moiety specific for visualizing each target nucleic acid, and the capture zone 108 comprises a specific capture moiety for capturing each target nucleic acid. The capture moieties for each of the different target nucleic acids are immobilized in distinct portions of the capture zone material.

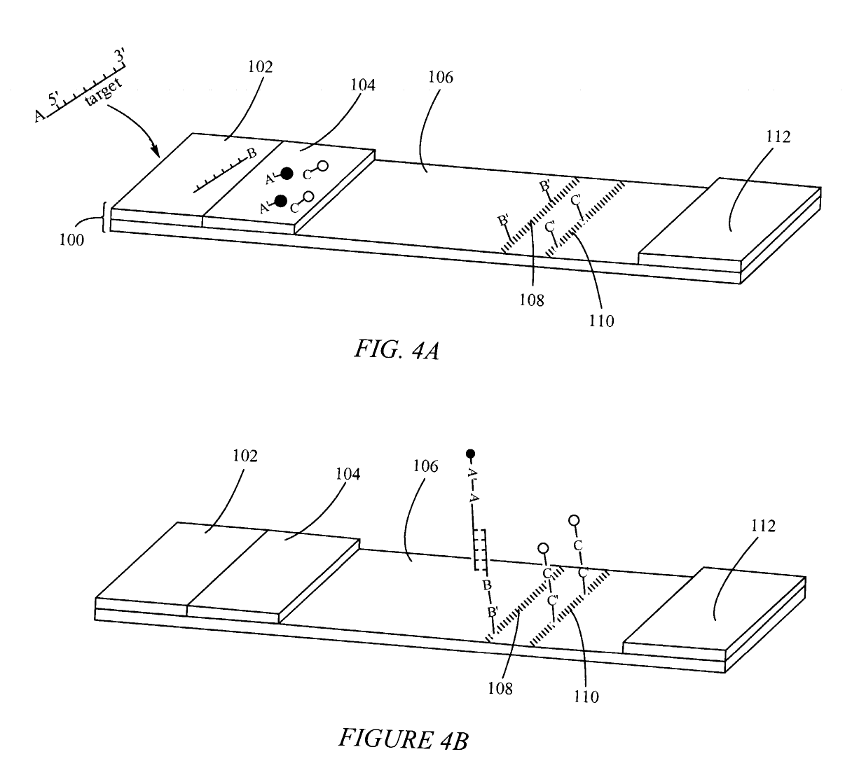

An alternate embodiment of an assay of the invention is illustrated in FIGS. 4A and 4B. The method illustrated in FIGS. 4A and 4B illustrates an embodiment wherein a labeled target nucleic acid, i.e., the target nucleic acid coupled to a binding partner (A) according to methods described herein, is detected using a lateral flow device comprising an oligonucleotide probe reversibly bound to the sample receiving zone material. Beginning with FIG. 4A, the assay device comprises test strip 100 having sample receiving zone 102. In this embodiment, sample receiving zone 102 comprises an oligonucleotide probe coupled to a second binding partner (B). Prior to applying the fluid sample containing the labeled target nucleic acid to the sample receiving zone 102, any nucleic acid present in the sample in a double stranded form is rendered single stranded by any denaturing method known in the art, and is subsequently taken up in a solution and applied directly to the sample receiving zone. Alternatively, the nucleic acid in the sample can be amplified prior to application to the sample receiving zone using any nucleic acid amplification method, such as those described herein. In this embodiment, the binding partner (A) can be coupled to the target nucleic acid during the amplification process. Test strip 100 also contains a first visible moiety reversibly bound to the labeling zone material 104 and coupled to ligand (A′). Ligand (A′) is designed to specifically recognize and bind to binding partner (A) coupled to the target nucleic acid. Test strip 100 further comprises capture zone 108 containing capture moiety (B′) immobilized on the capture zone membrane. Capture moiety (B′) is designed to specifically recognize and bind to binding partner (B) coupled to the oligonucleotide probe.

When the fluid sample containing the target nucleic acid is applied to the sample receiving zone, components contained in the fluid sample release the probes from the test strip and the target nucleic acid specifically hybridizes with the oligonucleotide probe to form a complex comprising first and second binding partners (A) and (B). The complexed target nucleic acid continues wicking along the test strip 100 by capillary action in the direction of the labeling zone 104.

As the fluid sample containing the complexed nucleic acid moves through the labeling zone 104, the visible moiety coupled to ligand (A′) is released from the test strip, binds to the binding partner (A) of the complexed nucleic acid, and flows with the complex in the direction of the capture zone 108 by virtue of being coupled to the complex as shown in FIG. 4B. Upon reaching the capture zone 108, binding partner (B) of the target nucleic acid/visible moiety is captured and immobilized in capture zone 108 by capture moiety (B′). Thus, if the target nucleic acid is present in the sample, the first visible moieties will be collected and bound in the capture zone 108 and form a visible signal, e.g., a colored line, which can be detected without having to contact the test strip with a visualizing reagent or chemical.

The assay outlined in FIGS. 4A and 4B can also incorporate the use of a control visible moiety. Thus, with reference to FIG. 4A, labeling zone 104 can further comprise a second (control) visible moiety reversibly bound to the labeling zone membrane and coupled to ligand (C), and capture zone 110 can comprise capture moiety (C′) immobilized to the capture zone membrane in region 110. During the assay illustrated in FIGS. 4A and 4B, the control visible moiety flows along with the fluid sample in the direction of the capture zone 110. Upon reaching the capture zone 110, binding partner (C) of the control visible moiety collect and are captured in capture zone 110 by capture moiety (C′), thus forming a visible, detectable signal, e.g., a colored line. As described, the control visible moieties may be the same or a different color than those used for binding to the binding partner of the complexed target nucleic acid. In an alternative embodiment, capture zones 108 and 110 overlap as described herein.

In another embodiment, the assay described with reference to FIGS. 4A and 4B can be used to detect two or more target nucleic acids. In this embodiment, the sample receiving zone 102 comprises an oligonucleotide probe specific for each target nucleic acid, the labeling zone 104 a specific and distinguishable first visible moiety specific for visualizing each target nucleic acid, and the capture zone 108 comprises a specific capture moiety for capturing each target nucleic acid. The capture moieties for each of the different target nucleic acids are immobilized in distinct portions of the capture zone material.

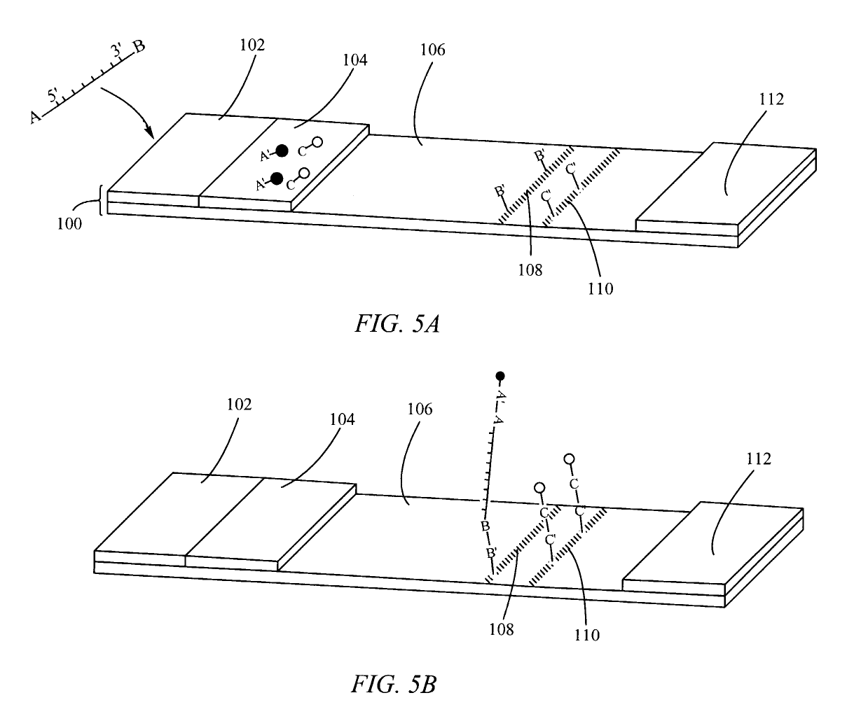

A third embodiment of an assay of the invention is illustrated in FIGS. 5A and 5B. The method illustrated in FIGS. 5A and 5B illustrates an embodiment wherein a doubly labeled target nucleic acid, i.e., a target nucleic acid which has been coupled to first and second binding partners (A) and (B) prior to the assay, is defected using a lateral flow device comprising an oligonucleotide probe reversibly bound to a membrane.

Beginning with FIG. 5A, in this third embodiment the assay device comprises test strip 100 having sample receiving zone 102 for receiving the fluid sample containing the doubly-labeled target nucleic acid. In this embodiment, the sample receiving zone does not contain any oligonucleotide probes. Test strip 100 also contains a first visible moiety reversibly bound to the labeling zone material 104 and coupled to ligand (A′). Ligand (A′) is designed to specifically recognize and bind to binding partner (A) coupled to the target nucleic acid. Test strip 100 further comprises capture zone 108 containing capture moiety (B′) immobilized to the capture zone membrane. Capture moiety (B′) is designed to specifically recognize and bind to binding partner (B) coupled to the target nucleic acid.

The doubly labeled target nucleic acid can be prepared by amplifying the target nucleic acid with a first and second primer comprising first and second binding partners, respectively, and then denaturing the amplified target nucleic acid to provide the single-stranded form. Alternatively, unamplified target nucleic acid can be labeled with a first and second label by known methods that do not involve amplification with labeled primers, such as the method described in Example 12. In either case, the target nucleic acid is applied to the sample receiving zone in a single-stranded form. When the fluid sample containing the doubly labeled target nucleic acid is applied to the sample receiving zone, the target nucleic acid wicks down the test strip 100 by capillary action in the direction of the labeling zone 104. As the doubly labeled target nucleic acid comprising binding partners (A) and (B) moves through the labeling zone 104, the ligand (A′) coupled to the first visible moiety binds to the binding partner (A) and thus flows with the target nucleic acid in the direction of the capture zone 108 by virtue of being coupled to the complex as shown in FIG. 5B. Upon reaching the capture zone 108, binding partner (B) of the target nucleic acid is captured and immobilized in capture zone 108 by capture moiety (B′). Thus, if the target nucleic acid is present in the sample, the first visible moieties collect and become bound in the capture zone 108, forming a visible signal, e.g., a colored line, which can be detected without having to contact the test strip with a visualizing reagent or chemical.

The assay outlined in FIGS. 5A and 5B can also incorporate the use of a control visible moiety. Thus, with reference to FIG. 5A, labeling zone 104 can further comprise a second (control) visible moiety reversibly bound to the labeling zone membrane and coupled to ligand (C), and capture zone 110 can comprise capture moiety (C′) immobilized to the capture zone membrane in region 110. During the assay illustrated in FIGS. 5A and 5B, the control visible moiety flows along with the fluid sample in the direction of the capture zone 110. Upon reaching the capture zone 110, binding partner (C) of the control visible moiety collect and are captured in capture zone 110 by capture moiety (C′), thus forming a visible, detectable signal, e.g., a colored line. As described, the control visible moieties may be the same or a different color than those used for binding to the binding partner of the complexed target nucleic acid. In an alternative embodiment, capture zones 108 and 110 overlap as described herein.

In another embodiment, the assay described with reference to FIGS. 5A and 5B can be used to detect two or more target nucleic acids. In this embodiment, the labeling zone 104 comprises a specific and distinguishable first visible moiety specific for visualizing each target nucleic acid, and the capture zone 108 comprises a specific capture moiety for capturing each target nucleic acid. The capture moieties for each of the different target nucleic acids are immobilized in distinct portions of the capture zone material.

The assays of the invention provide accurate and reliable results much faster than conventional methods. An assay of this invention typically provides a detectable signal within 10 to 300 seconds from commencement of the assay. Further, the assays and devices of this invention are able to provide direct detection of target nucleic acids without the need for amplification of the target nucleic acid prior to detection, provided that the sample contains the target nucleic acid in an amount that will provide a signal in the capture zone that can be detected with the naked eye.

The assays of this invention can be performed under high or low stringency conditions. The term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required when it is desired that nucleic acids which are not completely complementary to one another be hybridized or annealed together. Those skilled in the art know that numerous equivalent conditions can be employed to comprise low stringency conditions. Hybridization under stringent conditions requires a perfect or near perfect sequence match. Hybridization under relaxed conditions allows hybridization between sequences with less than 100% identity. Greater stringency can be achieved by reducing the salt concentration or increasing the temperature of the hybridization.

Thus, according to this invention, the term “stringency” refers to, but is not limited to: (1) the degree of annealing between an unlabed target nucleic acid and first and second oligonucleotide probes (FIG. 3A); (2) the degree of annealing between a singly labeled target nucleic acid and an oligonucleotide probe (FIG. 4A); (3) the degree of annealing during the labeling (coupling) of the target nucleic acid to a labeled primer to produce a singly labeled target nucleic acid (FIG. 4A); or (4) the degree of annealing during the labeling (coupling) of the target nucleic acid to a first and second labeled primer to produce a doubly labeled target nucleic acid (FIG. 5A).

In embodiments wherein the lateral flow device of this invention includes a heating sheet 116, an assay of this invention can be performed at temperatures above room temperature. Preferably, the assay is conducted at a temperature between about 25 and 95° C. Performing lateral flow assays at high temperatures is useful for a number of applications, including forensic medicine, and for determining Watson-Crick complementarity between nucleic acid strands.

This invention thus provides a complete, one-step, ready-to-use, fully functional lateral flow detection system for the detection of specific DNA or RNA targets. This construct contains all required reagents in an anhydrous format. This invention further provides a lateral flow device assembly which can be completely sealed in order to prevent amplicon or other nucleic acid contamination during use. In this embodiment, the integrity of the device is not compromised. In another approach, this invention demonstrates that direct detection of nucleic acids is possible without the need for amplification, a method which will facilitate faster detection of nucleic targets from various cells. In addition, the invention also relates to a method for performing nucleic acid testing where the temperatures are elevated an approach which will allow for stringency experiments useful in a variety of other applications such as forensic medicine. In addition to these advantages, the detection system described here will provide for a simple way to analyze nucleic acid targets by those not necessarily skilled in this art. Furthermore, it will facilitate the entry of true point-of-care for genomic analysis.

The assays and devices of the invention are applicable for the detection of any target nucleic acid. The term “target nucleic acid” refers to a nucleic acid targets to be detected by the devices and methods of this invention. Sources of target nucleic acids will typically be isolated from organisms and pathogens such as viruses and bacteria. Typical analytes may include nucleic acid fragments including DNA, RNA or synthetic analogs thereof Additionally, it is contemplated that targets may also be from synthetic sources. A target nucleic acid may incorporate one or more binding partners which may serve as members of a binding pair. Such binding partners are incorporated into the target nucleic acid in such a manner as to enable the binding partner to react with a second member of a binding pair. Binding partners may be coupled either at the 3′ end, the 5′ end or at any point between the 3′ and 5′ ends of the target nucleic acid. In one embodiment, the target nucleic acids are amplified as discussed below prior to analysis.

The term “nucleic acid” refers to an oligomer or polymer of nucleotides or mimetics thereof, as well as oligonucleotides having non-naturally-occurring portions which function similarly. It will be recognized by those skilled in the art that assays for a broad range of target nucleic acid sequences that may be present in a sample may be performed in accordance with the present invention. As used herein, the term “nucleotide” means either a deoxyribonucleotide or a ribonucleotide or any nucleotide analogue. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH₂, NHR, NR₂, or CN. RNAs may also comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

The assays and devices of the invention can detect a target nucleic acid obtained from a variety of samples. Thus, the term “sample” or “test sample” as used herein refers to any fluid sample potentially containing a target nucleic acid. Samples may include biological samples derived from agriculture sources, bacterial and viral sources, and from human or other animal sources, as well as other samples such as waste or drinking water, agricultural products, processed foodstuff, air, etc. Examples of biological samples include blood, stool, sputum, mucus, serum, urine, saliva, teardrop, tissues such as biopsy samples, histological tissue samples, and tissue culture products, agricultural products, waste or drinking water, foodstuff, air, etc. The present invention is useful for the detection of specific nucleic acid sequences corresponding to certain diseases or conditions such as genetic defects, as well as monitoring efficacy in the treatment of contagious diseases, but is not intended to be limited to these uses.

Amplification

The assays and devices of the invention are applicable for the detection of both unamplified and amplified target nucleic acids. As used in this invention, the term “amplification” refers to a process that results in an increase in the concentration (i.e., an increase in the copy umber) of a nucleic acid sequence relative to its initial concentration. Examples of amplification methodologies suitable for purposes of this invention include, but are not limited to, polymerase chain reaction (PCR), isothermal reactions such as nucleic acid sequence-based amplification (NASBA) (U.S. Pat. No. 5,130,238, specifically incorporated herein) or strand displacement amplification (SDA) (Walker, et al., PNAS 89:392, (1992), specifically incorporated herein by reference), ligase chain reaction, Qβ replicase (PCT publication WO 87/06270, specifically incorporated herein by reference), loop amplification (LAMP) (U.S. Pat. No. 6,410,278, specifically incorporated herein by reference), ramification amplification (RAM) (U.S. Pat. Nos. 5,876,924 and 5,942,391, specifically incorporated herein by reference), or cascade rolling circle amplification (CRCA) (U.S. Pat. Nos. 5,854,033 and 5,942,391, specifically incorporated herein by reference).

With amplification, certain specimens are inhibitory to the amplification reaction, providing false-negative results. To avoid this problem, a positive control, i.e., a control nucleic acid with primer recognition sequences attached to a totally irrelevant nucleic acid sequence, can be incorporated in the amplification step. This positive control primer is a component of the nucleic acid extraction reagents, thus controlling for sample extraction and delivery as well as detecting amplification failure. In one embodiment of the positive control is a lambda DNA sequence. The control nucleic acid is extracted and amplified along with the target nucleic acid and is detected by a line of immobile, coated microparticles on a detection membrane.

In certain embodiments of the invention, the target oligonucleotide primer and the control oligonucleotide primer used in the amplification steps of this invention contain a binding partner as a label which does not participate in the priming reaction. The binding partner is bound to at least one position of the oligonucleotide primer. For the derivatization of nucleic acid primers, various methods can be employed. See, Sambrook supra. The incorporation of the binding partner can take place enzymatically, chemically or photochemically. The binding partner can be derivatized directly to the 5′ end of the primer or contain a bridge 1 to 30 atoms long. In one embodiment, the bridge is linear. Alternatively, the bridge may comprise a branched chain with a binding partner molecule on at least one of the chain ends.

The present invention employs a variety of different enzymes, such as polymerases and ligases, to accomplish amplification of target nucleic acid sequences. Polymerases are defined by their function of incorporating nucleoside triphosphates to extend a 3′ hydroxyl terminus of a “primer molecule.” As used herein, a “primer” is an oligonucleotide that, when hybridized to a target nucleic acid molecule, possesses a 3′ hydroxyl terminus that can be extended by a polymerase and a hapten label at or near the 5′ terminus. Examples of polymerases that can be used in accordance with the methods described herein include, but are not limited to, E. coli DNA polymerase I, which is the large proteolytic fragment of E. coli polymerase I and is commonly known as “Klenow” polymerase, Taq-polymerase, T7 polymerase, T4 polymerase, T5 polymerase and reverse transcriptase. The general principles and conditions for amplification of nucleic acids using polymerase chain reaction are well known in the art.

Microparticle Selection

The visible moieties according to this invention are microparticles (i.e., a micrometer-sized particles) that can be directly visualized, such as a dyed particle. Any suitable insoluble particle may be employed for purposes of this invention, including, but not limited to, particles of a polymeric material which may include, but is not limited to, a thermoplastic (e.g., one or more of polystyrenes, polyvinyl chloride, polyacrylate, nylon, substituted styrenes, polyamides, polycarbonate, polymethylacrylic acids, polyaldehydes, and the like), latex, acrylic, latex or other support materials such as silica, agarose, glass, polyacrylamides, polymethyl methacrylates, carboxylate modified latex, Sepharose, methacrylate, acrylonitrile, polybutadiene, metals, metal oxides and their derivatives, silicates, paramagnetic particles and colloidal gold, dextran, cellulose, and liposomes, and natural particles such as red blood cells, pollens, and bacteria. The size of the microparticles used in this invention is selected to optimize the binding and detection of the labeled target nucleic acid, and are typically 0.01 to 10.0 μm in diameter and preferably 0.01 to 1.0 μm in diameter, specifically not excluding the use of either larger or smaller microparticles as appropriately determined. In one embodiment, the microparticle is substantially spherical in shape. The preferred microparticle in the present invention is composed of latex containing a colored dye.

In accordance with the invention, the microparticles are coated with ligand (i.e., a binding partner) specific for a binding partner that is coupled to or complexed with a target nucleic acid. Methods of coupling ligands to particles are well known in the art. For example, in one embodiment, the microparticles possess surface sulfate charge groups that can be modified by the introduction of functional groups such as hydroxyl, carboxyl, amine and carboxylate groups. The functional groups are used to bind a wide variety of ligands (binding partners) to the microparticles, and are selected based on their ability to facilitate binding with the selected ligand. Conjugation of the ligands to the microparticle is accomplished by covalent binding or, in appropriate cases, by adsorption of the ligand onto the surface of the microparticle. Techniques for adsorption or covalent binding of receptors to microparticles are well know in the art and require no further explanation. The preferred method of attachment of the ligand to the microparticles is covalent binding.

Self-Contained Devices

The present invention further provides novel self-contained devices for detecting a target nucleic acid sequence that is present in a sample. The self-contained devices disclosed herein eliminate the possibility of cross-contamination from one sample to another by integrating nucleic acid extraction, amplification, and detection strategies in completely enclosed, disposable devices. In general, a self-contained device of this invention comprises a plurality of separate, sequential chambers, for example, an extraction chamber, a waste chamber, an amplification chamber, and a detection chamber, wherein a sample which may contain the target nucleic acid to be detected is extracted, amplified and detected in separate and sequential chambers. To use a multi-chambered self-contained device of this invention, a sample containing target nucleic acid and control nucleic acid is introduced into an extraction chamber for extraction of nucleic acid. The extraction chamber incorporates a nucleic acid extraction/solid phase nucleic acid binding protocol providing a rapid method of nucleic acid purification. The preferred extraction method makes use of chaotropic agents such as guanidine thiocyanate (GuSCN) to disrupt the cell membranes and extract the nucleic acid. Proteins are degraded by proteinases. The extracted nucleic acid binds to a solid phase membrane in the extraction chamber. The design of a fitting between the solid phase membrane and a seal located directly below the solid phase prevents waste from entering the amplification chamber.

In one embodiment, after the sample has been added to the extraction chamber, a supply assembly unit locks onto the top of a processor assembly unit by connecting a first and a second fitting. Following a 10-15 minute incubation allowing for nucleic acid extraction, the first of four plungers is depressed. Air in a compartment forces the extraction mixture past the solid phase membrane binding the nucleic acid. The filtrate is collected in a waste chamber. Depression of a second plunger forces a wash buffer stored in a wash buffer compartment across the solid phase membrane and filtrate passes to the waste chamber. The seal located directly below the solid phase membrane is disposed at an angle to aid in efficient collection of the waste. Depression of a third plunger forces air stored in a compartment across the solid phase membrane, insuring that all of the wash buffer is removed. The processor assembly unit twists, simultaneously breaking the seal and closing off a waste chamber conduit. Depression of a fourth plunger delivers an elution buffer stored in a compartment for elution of the nucleic acid from the solid phase and delivers a volume of nucleic acid into an amplification chamber.

The amplification chamber contains the reagents for amplification and hybridization. In an alternative embodiment, reagents for amplification and hybridization are in separate chambers. The amplification/hybridization process is characterized in that the sample is treated, after extraction, with two distinct labeled oligonucleotides primers. The sequence of the first primer is complementary to a partial sequence of the target nucleic acid and is labeled with hapten, for example, biotin. The sequence of the second primer is complementary to a partial sequence of the control nucleic acid and labeled with a second hapten, for example, digoxigenin. Either primer may contain a promoter region. Subjecting the mixture to amplification, preferably isothermal amplification, results in hapten labeled target nucleic acid sequences and hapten control nucleic acid sequences. The labeled, amplified nucleic acid sequences hybridize to oligonucleotides which are conjugated to microparticles of suitable color and diameter for detection. The microparticles are conjugated either with an oligonucleotide specific for binding a nucleic acid sequence on the target or with an oligonucleotide specific for binding a nucleic acid sequence on the control nucleic acid. The resulting microparticles, bound by hybridization to the amplicons, are detected in the detection chamber.

1. Three-Chambered Self-Contained Device

One embodiment of a self-contained device of the present invention, generally illustrated in FIG. 6, comprises a first hollow elongated cylinder with a single closed end and a plurality of chambers therein, and a second hollow elongated cylinder positioned contiguously inside the first cylinder and capable of relative rotation. In this embodiment, the extraction and amplification of nucleic acids take place in the second cylinder (the reaction chamber) of the self-contained device, detection takes place in a detection chamber of the first cylinder, and collection of waste occurs in a waste chamber of the first cylinder. The chambers of the self-contained device of FIG. 6 are functionally distinct, sequential and compact. The chambers deliver precise volumes, dispense reagents and collect waste. All of the steps of nucleic acid extraction, amplification and detection occur in the completely self-contained device with simple, fool-proof directions for use as described below.

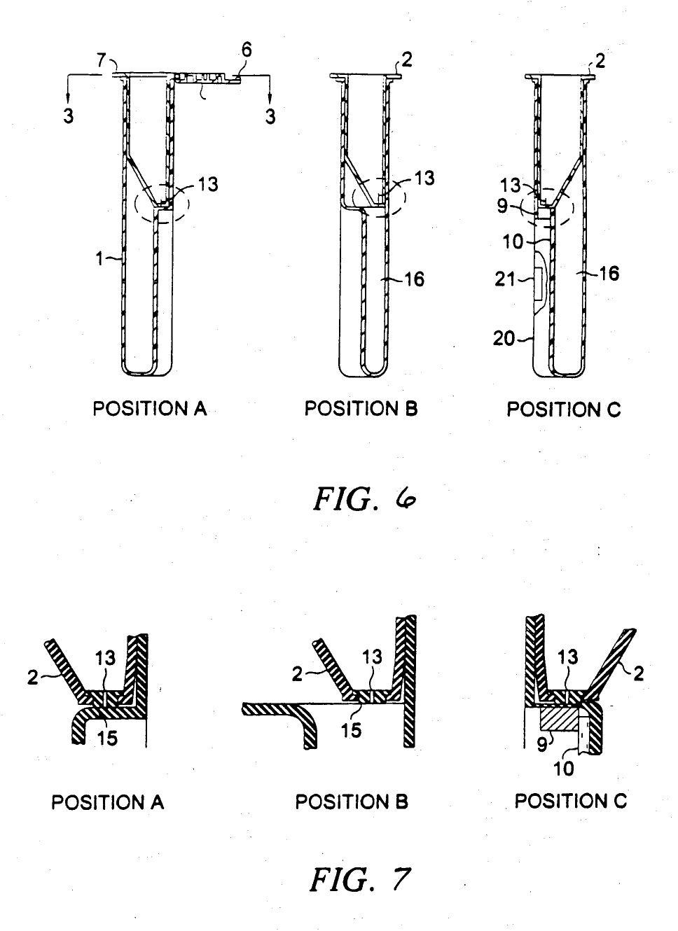

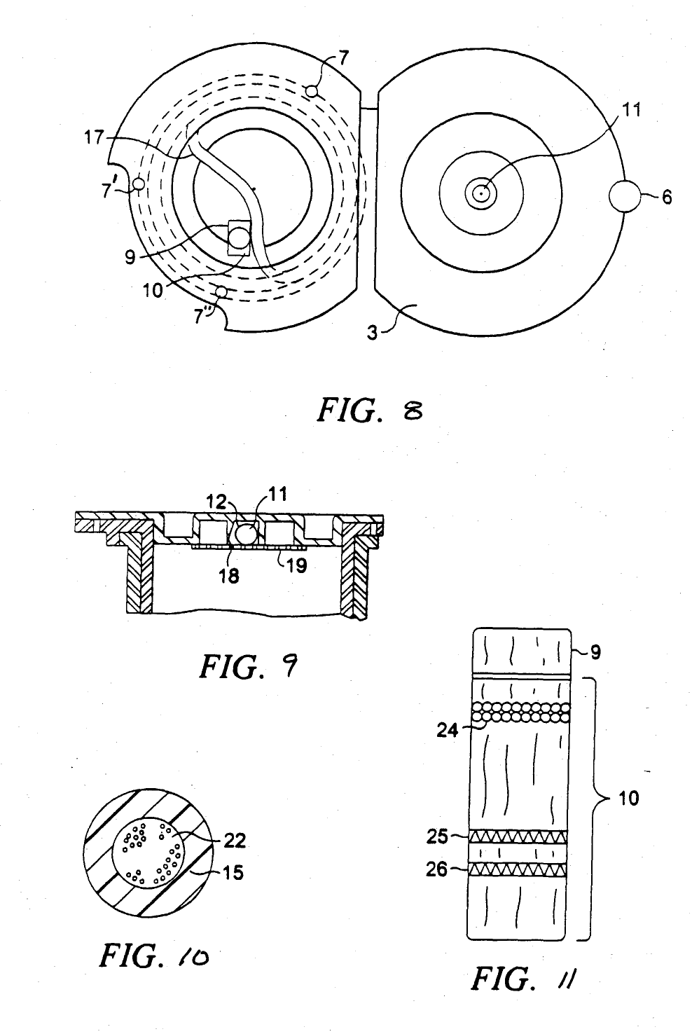

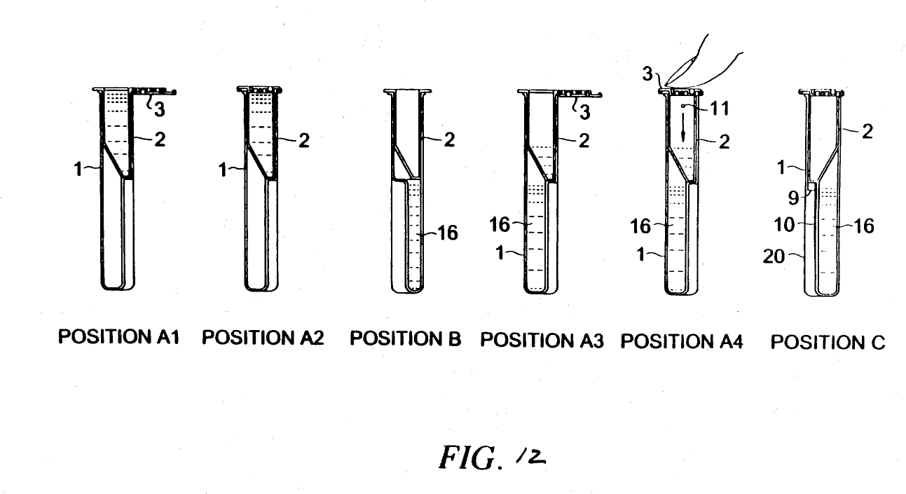

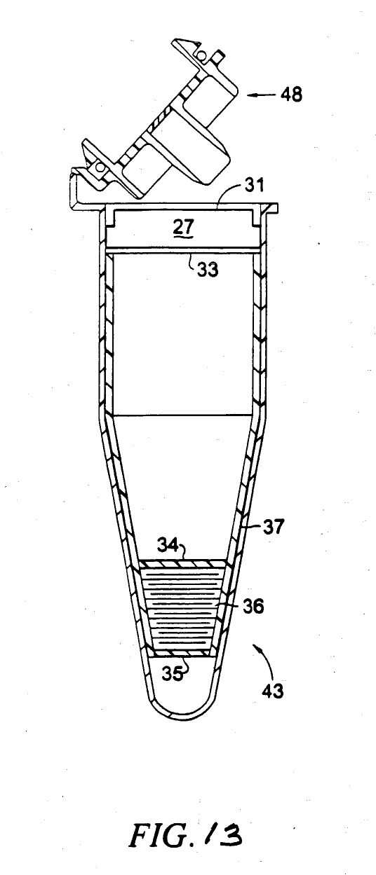

With continued reference to FIG. 6, one embodiment of a self-contained device of this invention comprises a first hollow elongated cylinder 1 having one closed end and an integrally-molded cover 3 hinged to the opposing, opened end, and a second hollow elongated cylinder 2 that is positioned contiguously inside the first cylinder 1 and is capable of relative rotation. The preferred embodiment of the second cylinder 2 is a tapered cylinder terminating with an aperture 13 having a sealing lip 15 as shown in FIG. 7. The first cylinder 1 further consists of two chambers: a reservoir or waste chamber 16 and a detection chamber 20, the detection chamber further comprising a pad 9 and a strip 10. When sample is introduced into the device, nucleic acid extraction and amplification takes place in the second cylinder 2. The first hollow elongated cylinder 1 contains the detection chamber 20 having a means for detection and reservoir 16 for collecting the lysis buffer used in the extraction process and other buffers used in subsequent washes.

The second cylinder 2 rotates relative to the first cylinder 1 and locks into position A, position B or position C. At the tapered end of the second cylinder 2, an aperture 13 having a sealing lip 15 enables the second cylinder 2 to engage with either the detection chamber 20 or reservoir 16 of the first cylinder 1. The hinged cover 3 has one indexing pin 6 shown in FIG. 6, position A) used for locking the second cylinder 2 in positions A, B and C. The second cylinder 2 contains three notches 7, 7′ and 7″ for indexing with the indexing pin 6 and locking the relative rotation of cylinders 1 and 2. The second cylinder 2 is closed to the reservoir 16 in the closed position A. In position A, the second cylinder 2 is sealed, allowing for the extraction step and the amplification step to take place. For purposes of illustration only, the method of using the self-contained device of FIG. 6 will be discussed with respect to amplification methods that produce bifunctionally labeled, amplified nucleic acids. However, it will be understood that other amplification methods, such as those that produce singly-labeled nucleic acids or unlabeled nucleic acids, may be used in the self-contained device of FIG. 6, as discussed below in detail. Thus, in one embodiment, the amplification produces a bifunctionally labeled target nucleic acid having a hapten A on one end and a hapten B on the other end of the amplified target nucleic acid. Amplification also produces a bifuntionally labeled control nucleic acid having a hapten C on one end and a hapten D on the other end.

In open position B, the second cylinder 2 is such that the opening 13 in the second cylinder 2 is not sealed and is over the reservoir 16. In open position B, the second cylinder 2 allows flow to the reservoir 16.

In elute position C, the second cylinder 2 is rotated such that the second cylinder 2 is not sealed and the opening 13 is over an absorbent pad 9 located in the detection chamber 20. In elute position C, amplified nucleic acid target and control are able to wick into the detection chamber 20. The absorbent pad 9 collects the amplified product and wicks the product onto a strip 10 of nylon, nitrocellulose or other suitable material. The strip 10 contains colored microparticles 24 and capture zones 25 and 26 for the target and the control sequences, respectively (FIG. 11). The detection chamber 20 contains a transparent viewing window 21 for observing the results of the reaction.

FIG. 7, which shows enlargements of the encircled portions of FIG. 6, illustrates the preferred embodiment of the sealing mechanism of the self-contained device of FIG. 6. In closed position A, the second cylinder 2 is sealed by a sealing lip 15 at the bottom of cylinder 2. The sealing lip 15 is composed of a flexible material that can be compressed when in contact with a solid surface 17 (FIG. 8) at the top of the first cylinder 1. With continued reference to FIG. 7, in open position B, rotation of the second cylinder 2 relative to the first cylinder 1 allows the contents of the second cylinder 2 to flow into the reservoir 16 through a solid phase 22 (FIG. 10), for example a porous membrane, in the bottom of the second cylinder 2. In this position, the sealing lip 15 is extended beyond the plane of compression and allows fluid to flow into the reservoir 16. The second cylinder 2 can also be rotated relative to the first cylinder 1 into elute position C. In this position, the sealing lip 15 is again extended beyond the plane of compression and allows amplified nucleic acid and control nucleic acid to wick onto an absorbent pad 9 and a strip 10 of membrane used for the detection step.

A top plan view of the self-contained device of FIG. 6 and the hinged cover 3 in the open position is illustrated in FIG. 8. The index pin 6 is located on the hinged cover 3. Three index notches 7, 7′, and 7″ are located on the second cylinder 2. The hinged cover 3 contains a reaction bead 11 within a reaction bead chamber 12 (FIG. 9). This bead 11 contains the reaction enzymes and other reagents required for the amplification step. The hinged cover 3 may also contain a knife-edge 18, which when sufficient pressure is applied punctures a foil membrane 19 (FIG. 9), releasing the reaction bead 11 into the second cylinder 2.

A cross-section of the bottom of the second cylinder 2 is illustrated in FIG. 10. The sealing lip 15 contains a solid phase 22 (e.g., a porous membrane) that binds the extracted nucleic acids or a solid phase 22 that holds a silica slurry (not shown) in the second cylinder 2.

As stated above, detection takes place in detection chamber 20. Preferably the detection method is a lateral flow assay. The specific reagents in the detection chamber will depend on the type of amplified product produced, that is whether the amplification produces a bifunctionally labeled, singly labeled, or unlabeled nucleic acid. In one embodiment, detection chamber 20 of the first cylinder 1 contains a pad 9 and a strip 10. FIG. 11 illustrates strip 10 containing a region with immobilized colored microparticles 24 and two capture zones 25 and 26. In this embodiment, the microparticles 24 are coated either with a receptor (A′) that is specific to hapten A the target nucleic acid, or with a receptor (C′) that is specific to hapten (C) on the control nucleic acid. Additionally, the target sequence capture zone 25 contains receptors B′ that are specific for hapten (B) on the target sequence, and control sequence capture zone 26 contains receptors (D′) that are specific for hapten (D) on the control sequence.

FIG. 12 depicts the sequence of steps for the extraction, amplification and detection of nucleic acid sequences using the embodiment of the self-contained device illustrated in FIG. 6. In the closed position (A1), a sample containing a control nucleic acid and the target nucleic acid to be detected (if present) is introduced into the second cylinder 2. Preferably, second cylinder 2 has a capacity of 0.001 to 25 mL. The second cylinder 2 contains dry lysing reagents for extraction of nucleic acids. The sample provides the liquid that resuspends the lysing reagents. After a brief incubation period with the cover 3 closed (position A2), the second cylinder 2 is rotated into open position (B). The extracted nucleic acid remains bound to the solid phase 22 or the silica slurry (not shown) in the second cylinder 2, while the liquid flows into the reservoir 16. In open position B, several washes with buffer or water follow.

Next, the second cylinder 2 is rotated into closed position A3 such that the second cylinder 2 is sealed. Water is added to the second cylinder 2 and the hinged cover 3 is closed (position A4). When sufficient pressure is applied to the hinged cover 3 as shown in position A4, foil membrane 19 is punctured by knife-edge 18 (FIG. 9), and the reaction bead 11 is released from the reaction bead chamber 12 into the second cylinder 2. The reaction bead 11 carries the enzymes necessary for amplification, which are resuspended in the Water. Amplification takes place on the solid phase 22 (FIG. 10) or silica slurry (not shown) containing the bound, extracted nucleic acids and produces bifunctionally labeled amplified target nucleic acid labeled with haptens A and B, and bifunctionally labeled control nucleic acid labeled with haptens C and D.

After an appropriate incubation period, the second cylinder 2 is rotated relative to the first cylinder 1 into elute position (C). The amplification reaction mixture is able to enter the detection chamber 20 as it is absorbed onto the pad 9. When the pad 9 absorbs a sufficient amount of liquid, the reaction mixture is wicked onto the membrane strip 10. On the membrane strip 10, receptors (A′) on colored microparticles 24 bind to haptens A on the amplified target, and receptors (C′) on microparticles 24 bind to haptens (C) on the control nucleic acids, and microparticle-bound nucleic acids travel to the capture zones 24 and 25 on the membrane strip 10. The target capture zone 25 contains receptors (B′) specific for haptens (B) on the target sequence, and control capture zone 26 contains receptors (D′) specific for haptens (D) on the control sequence. A visible line of detection forms at capture zone 25 if the target sequence is present and at capture zone 24 for the control sequence. The lines of detection are viewed from the transparent viewing window 21 (FIG. 6).

The bulk of the device shown in FIG. 6 is composed of a material that does not facilitate binding of nucleic acids and proteins. The preferred material is heat and cold resistant material which is light weight, rigid and sturdy. The preferred size is compact enough to fit into conventional size heat blocks, however, the size may be scaled up or down, accordingly. In a preferred embodiment, the self-contained device of FIG. 6 is inserted into a constant temperature environment such as a heat block, allowing the reactions to proceed at the preferred conditions of constant temperature.

2. Self-Contained Device Comprising a Matrix Tube

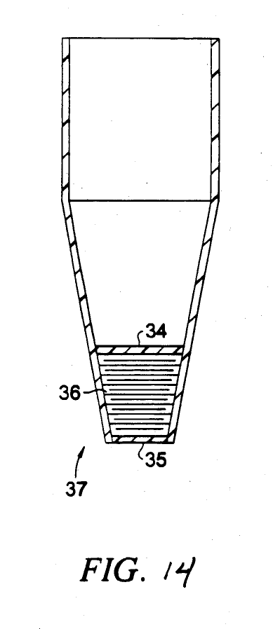

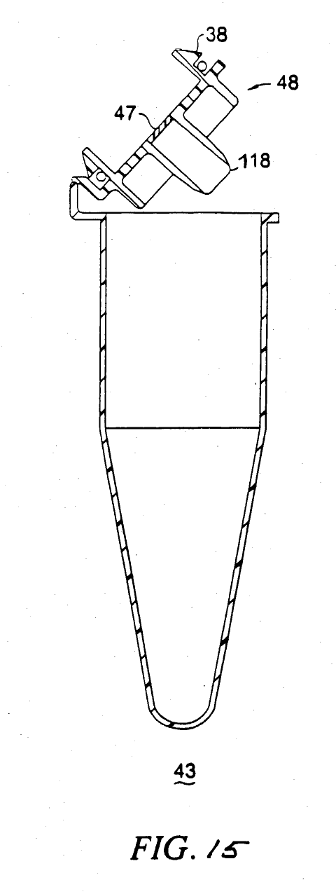

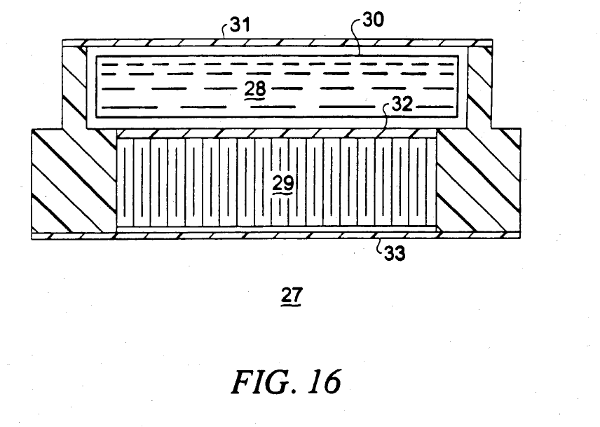

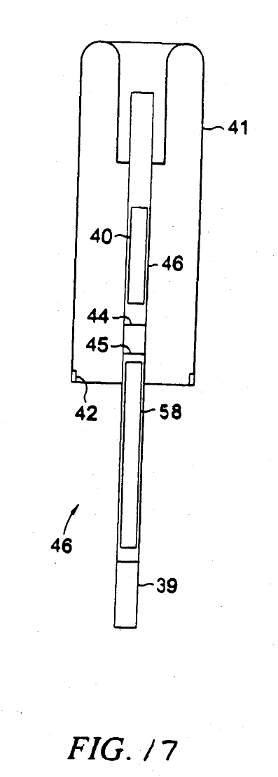

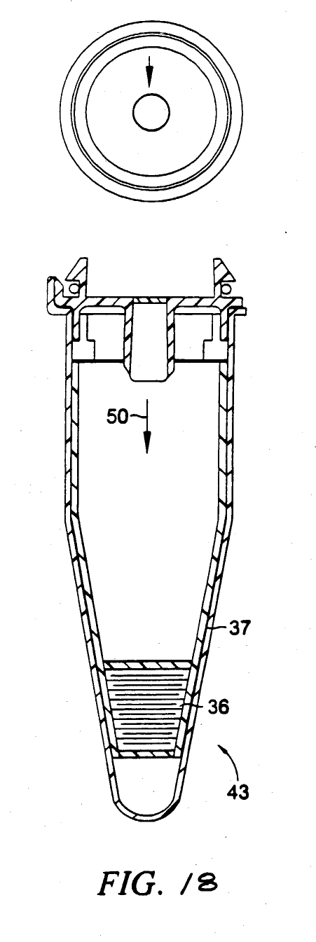

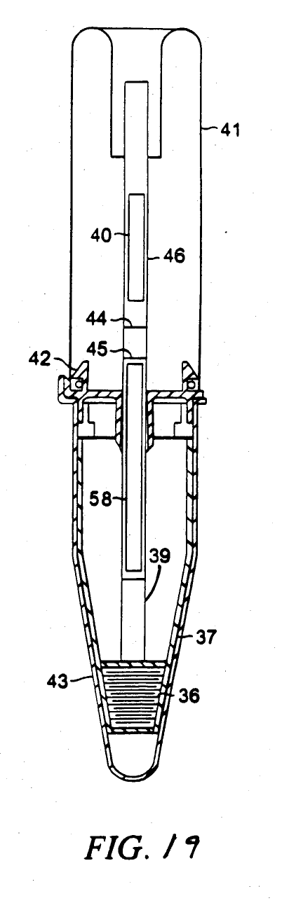

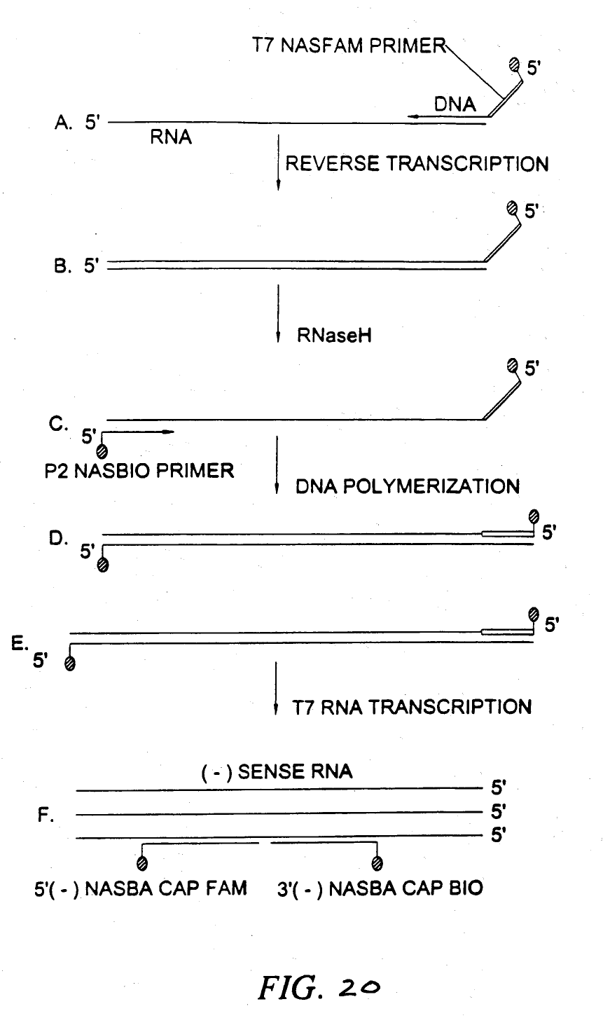

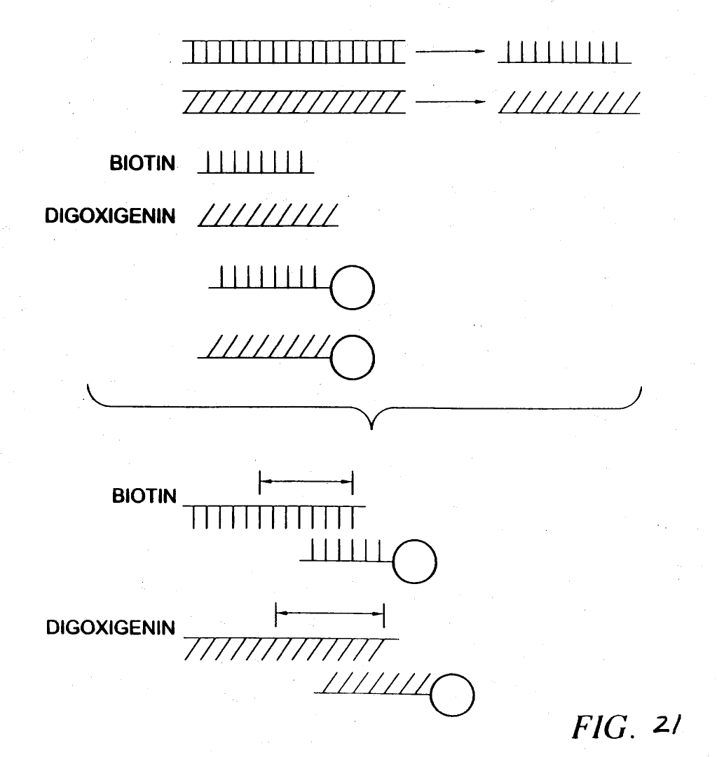

Yet another embodiment of a self-contained device of the invention is illustrated in FIG. 13 and includes a self-contained integrated particle assay device for use with polymerase chain reaction (PCR). This embodiment is defined by a matrix tube 37 (FIG. 14), a PCR tube 43 (FIG. 15), a reagent or reagents 29 which may be contained in a reagent cell 27 (FIG. 16), and a result stick 46 (FIG. 17). The reagent cell 27 (FIG. 16) is further defined by two pouches or chambers: a first pouch 30 containing liquid 28 such as water or other appropriate diluent, and a second pouch containing lyophilized PCR reagents 29. Alternatively, the second pouch may contain a lyophilized reagent bead or beads. Three foil seals, an upper 31, middle 32 and a lower 33 (FIG. 16), are disposed and positioned within the reagent cell 27 such that they separate and contain the liquid 28 and the PCR reagents 29.

PCR reagents 29 include, for example, specific primers for target nucleic acid and control nucleic acid, enzymes, stabilizers, and buffers useful for PCR amplification of target and control molecules. At least two of the target specific primers are labeled with distinct haptens (A) and (B), and at least two of the primers for the control nucleic acid sequence are labeled with distinct haptens (C) and (D). These haptens are incorporated into the target and control amplification products (“bifunctional haptenization”) during the amplification reaction.

In one embodiment of the self-contained device of FIG. 13, matrix tube 37 (FIG. 14) comprises an upper screen 34 and lower screen 35 between which a solid phase matrix 36 specific for nucleic acid binding is sandwiched. In an alternate embodiment (not shown) of the self-contained device of FIG. 13, the solid phase matrix 36 is directly adhered or bound directly to the interior wall of the matrix tube. Thus, it is not a necessary or defining facet of the instant invention that the solid phase matrix 36 be sandwiched between an upper screen 34 and a lower screen 35 as shown in FIG. 14. The solid phase matrix 36 comprises, for example, aluminum oxide or silicon dioxide. The top of the matrix tube 37 may snap fit with a mating and locking connection mechanism, such as a Luer-lock type. The matrix tube 37 is constructed from any material suitable for facilitating thermo-regulation and fluid transfer, such as thin wall or porous plastic. The general shape of matrix tube 37 is that of what is generally known as either a PCR or Eppendorf tube, i.e., a conical-shaped tube having a closing top portion and configured in size such that it is able to be contiguously disposed within the PCR tube 43 of the instant device.

Moving now to FIG. 15, the PCR tube 43 is a tube generally accepted in the art as a PCR tube and further contains a foil, plastic, rubber or other elastomer patch 47 disposed on the interior of its lid 48. This patch 47 seals the area through which the result stick 46 (FIG. 17) passes upon its introduction therethrough, after the PCR reaction is complete. The lid 48 may contain a sharp knife-like piercing feature 118 able to pierce all three of the foil seals 31, 32, and 33 of the reagent cell 27 (FIGS. 13 and 16), thus resuspending the reagents 29 in the liquid 28. The PCR tube 43 further contains a locking and/or sealing means 38 within lid 48 that, in turn, seals the entry aperture created upon introduction of the result stick 46 into to the PCR tube 43. For example, the locking or sealing means may include foil, plastic, rubber or other elastomer.

Referring now to FIG. 17, the result stick 46 consists of an elongated transparent body 41, for example plastic or polycarbonate, having a top portion intended for handling the result stick 46 and a bottom portion intended for detection. A snap fit type seal 42 locks the result stick 46 into the PCR tube 43. Moving from the bottom to top portion of the result stick 46, there is disposed thereon an absorbent sample pad 39, a solid phase matrix 58, for example a porous membrane, and a waste pad 40, respectively. The absorbent sample pad 39 is comprised of any generally accepted material suitable for lateral flow and dip stick type assays. The pad 39 is fabricated to contain microparticles conjugated with a receptor specific for hapten A, as well as microparticles conjugated with a receptor specific for hapten C. Alternatively, the microparticles may be on the porous membrane 58 itself. The porous membrane 58 further carries a control indicator line 44 and a sample detection indicator line 45 that have been strategically applied and dried thereon. The sample detection indicator line 45 consists of a receptor specific for hapten B. The control detection indicator line 44 consists of a receptor specific for hapten D.

The operating sequence of the embodiment of the self-contained device illustrated in FIGS. 13-19 entails adding a sample containing the target nucleic acid (if present) and a control nucleic acid in lysis buffer to the matrix tube 37 directly or through a suitable vessel. A suitable vessel may include, for example, a syringe that snap fits onto the matrix tube 37 via a mating and locking connection system. After denaturization, the sample passes through the matrix tube 37 into a waste area, and the target and control nucleic acids bind specifically to the solid phase matrix 36. The sample passes through the tube via, for example, gravity flow or any suitably adaptable method, such as vacuum controlled flow. Next, the matrix-bound nucleic acids are washed with suitable buffer and the matrix tube 37 is placed into the PCR tube 43 (FIG. 13). The reagent cell 27 is inserted into the PCR tube 43 as illustrated in FIG. 13. By pushing firmly on the cap 48 of the PCR tube 43 the foil seals 31, 32 (not shown) and 33 of reaction cell 27 are pierced, thus causing reagent 29 (not shown) to be resuspended in liquid 28 (not shown). The liquid resuspension drops to the bottom of the matrix tube 37 and PCR tube 43 as shown by arrow 50 in FIG. 18 and enters the solid phase matrix 36. Reaction volume is calculated to be sufficient such that the solid phase matrix 36 lies below the meniscus created by the reaction reagents. The PCR tube 43 (FIG. 18) containing the matrix tube 37, resuspended reagents 29 and nucleic acid bound to the solid phase matrix 36 is then inserted into a thermocycler for amplification of the target and control sequences. In one embodiment, the amplification produces bifunctionally labeled target nucleic acids labeled with haptens (A) and (B), and bifunctionally labeled control nucleic acids labeled with haptens (C) and (D).

Upon completion of the PCR event, the device is removed from the thermocycler and the result stick 46 is inserted into the PCR tube 43 through the foil patch 47 in the lid 48 (FIG. 19). The absorbent sample pad 39 of the result stick 46 comes into contact with the aqueous reaction mixture containing amplified target nucleic acid (if target was present in the sample) and amplified control nucleic acid. The mixture soaks into or wicks up the absorbent sample pad 39 where the microparticles coated with either receptors (A′) or (C′) bind to their respective haptens. That is, microparticles coated with receptors (A′) bind to haptens (A) on-target nucleic acids, and microparticles coated with receptors (C′) bind to haptens (C) on control nucleic acids. Once the absorbent pad 39 is saturated, the reaction mixture and the nucleic acid-bound microparticles wick up the porous membrane 58 via capillary flow toward the control and sample detection indicator lines 44 and 45, respectively. Wicking is facilitated by the presence of the waste pad 40. If the target nucleic acid is present, hapten (B) on the microparticle-bound target nucleic acid binds to a receptor (B′) contained in the target detection indicator line 45, forming a visible line of detection. Also, haptens (D) on the microparticle-bound control sequences bind to receptors (D′) contained in the control detection indicator line 44, forming a visible line. The detection results are viewed through the transparent body 41 of the result stick 46.

The self-contained devices disclosed herein provide for extremely rapid, economical nucleic acid detection. Further, the self-contained devices significantly reduce the risk of cross contamination in that neither amplification reagents nor amplicons are manipulated. Elimination of cross contamination opens the door to mass screening including automation.

The self-contained devices of the present invention can be used in the diagnoses of infectious diseases of genetic, bacterial or viral origin. The high sensitivity of analysis using the self-contained devices of this invention allows for the early detection of disease and an opportunity for early treatment. Analysis by this invention may monitor the efficacy of treatment, for example, to monitor HIV virus in the plasma of patients undergoing therapy. The low complexity of the device lends itself to “point of care” testing in clinics and physician's offices. The portability of the device provides for “on site” analysis to detect nucleic acid sequences in the areas of forensics, agriculture, environment and the food industry.

The cost of nucleic acid analysis using the self-contained devices of this invention is significantly less than other methods currently in use to detect amplified nucleic acids. The time frame for detecting an amplified sequence is reduced drastically. There is no danger from potentially hazardous chemicals. The analysis does not require special waste disposal procedures. The requirements of many washes in an immunometric or hybridization approach are eliminated. The self-contained device does not require special equipment, other than a standard, constant temperature heat block.

The following examples serve to explain and illustrate the present invention. The examples are not to be construed as limiting of the invention in anyway. Various modifications are possible within the scope of the invention.

### EXAMPLE 1

### Isothermal Amplification Approach to Detection with Labeled Amplified Target Sequence Using NASBA

One amplification methodology for use in this invention is an isothermal reaction such as nucleic acid sequence-based assay (NASBA). The primary product of the NASBA reaction is single strand RNA. The NASBA reaction utilizes a primer containing a T7 polymerase promoter. Following T7 transcription, up to 100 copies of target RNA are produced. These copies are the same sequence as the original target RNA. They serve as templates, thus starting the cycle again and resulting in up to a billion fold amplification of the original template.

In order to incorporate NASBA into the devices disclosed herein, probes that allow the formation of a bifunctionally haptenized amplification product have been designed. For NASBA there are two possible strategies: 1) design amplification primers that are haptenized; and 2) use two haptenized capture oligonucleotides which bind to the product RNA. The model system chosen is to the HIV POL gene.

The first strategy using NASBA haptenization, i.e., the design of amplification primers that are haptenized, is illustrated in FIG. 20, steps A-D. A T7 NASFAM haptenization primer, containing a T7 transcriptase promoter and an attached fluorescein, binds to the target RNA (FIG. 20, step A). A reverse transcriptase transcribes a DNA copy of the RNA, as illustrated in step B of FIG. 20. The original RNA strand is digested by RNase H. A reverse haptenization primer, P2 NASBIO with attached biotin, binds to the antisense DNA (FIG. 20, step C) and is extended by the DNA polymerase activity of the reverse transcriptase.

The haptenized primers are as follows:

| | |
|---|---|
| T7 NASFAM (T7-promoter primer): | |
| 5′gluorescein-AATTCTAATACGACTCACTATAGGGTGCTATGTCACTTCCCCTTGGTTCTCT-3′ | SEQ ID NO:1 |
| P2 NASBIO (reverse primer): | |
| 5′-biotin-AGTGGGGGGACATCAAGCAGCCATGCAAA-3′ | SEQ ID NO:2 |

The resulting double-stranded bi-haptenized DNA intermediate, containing a biotin label at one end and a fluorescein label at the other end, is illustrated in step D of FIG. 20. This complex gives signal in lateral flow or slide agglutination assays.

The second strategy for using NASBA in this invention, i.e., the use of two haptenized oligonucleotides which bind to the product RNA, is illustrated in FIG. 20, steps E-F. T7 RNA polymerase binds to the promoter region (step E) to manufacture many copies of a minus-sense RNA, as shown in steps E and F of FIG. 20. This RNA contributes to the manufacture of the DNA intermediate by similar means. Two capture oligonucleotides, each having one hapten of either fluorescein or biotin, bind to the minus-sense RNAs (FIG. 20, step F) giving bifunctional haptenized complexes. These complexes give signal in lateral flow or slide agglutination. The haptenized capture oligonucleotides, designed to bind to the minus-sense RNA product are:

| | |
|---|---|
| 5′-NASBA CAP FAM: | |
| 5′-fluorescein-TGGCCTGGTGCAATAGGCCC-3′ | SEQ ID NO:3 |
| 3′-NASBA CAP-BIO: | |
| 5′-CCCATTCTGCAGCTTCCTCA-biotin-3′ | SEQ ID NO:4 |

### EXAMPLE 2

### Isothermal Amplification Approach to Detection with Bifunctionally Labeled Amplified Target Sequence Using Strand Displacement Amplification

The instant strand displacement amplification (SDA) is another example of an isothermal amplification methodology that can be detected in the self-contained devices of this invention by using microparticles and bifunctionally labeled product.

SDA technology is described in U.S. Pat. No. 5,455,166, which is specifically incorporated herein. SDA is isothermal amplification based on the ability of a restriction enzyme to nick the unmodified strand of a hemiphosphorothioate from its recognition site and the ability of DNA polymerase to initiate replication at the nick and displace the downstream non-template strand. Primers containing recognition sites for the nicking restriction enzyme bind to opposite strands of target DNA at positions flanking the sequence to be amplified. The target fragment is exponentially amplified by coupling sense and antisense reactions in which strands displaced from the sense reaction serve as a target for the antisense reaction and Vice versa.

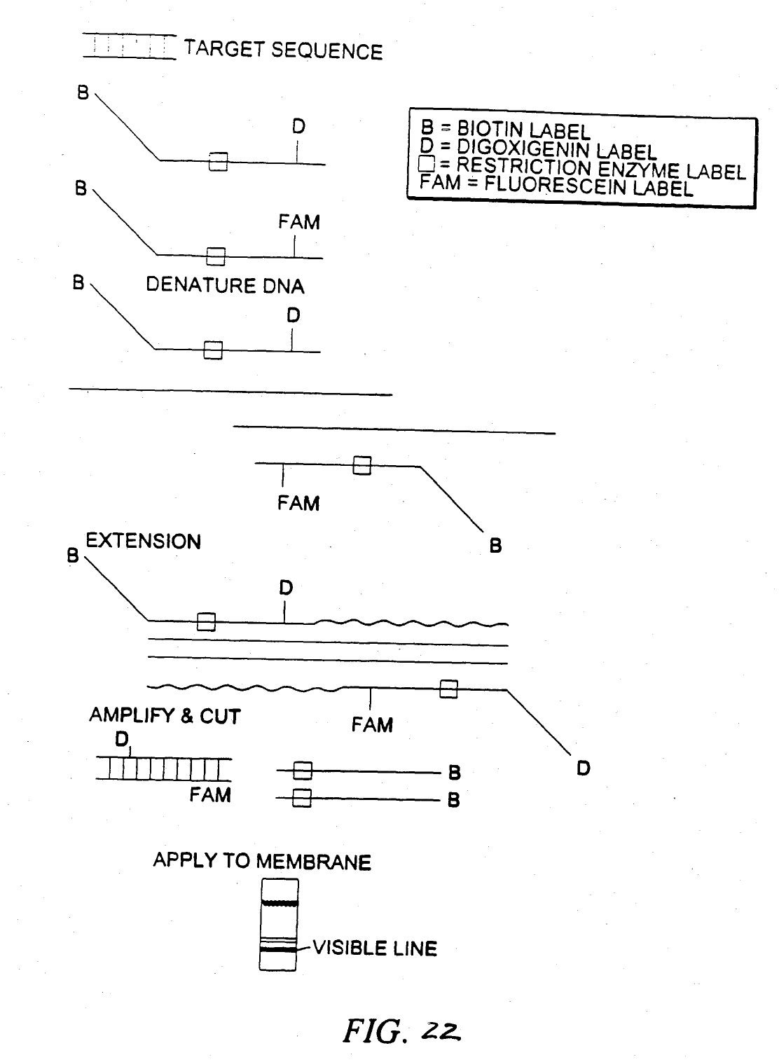

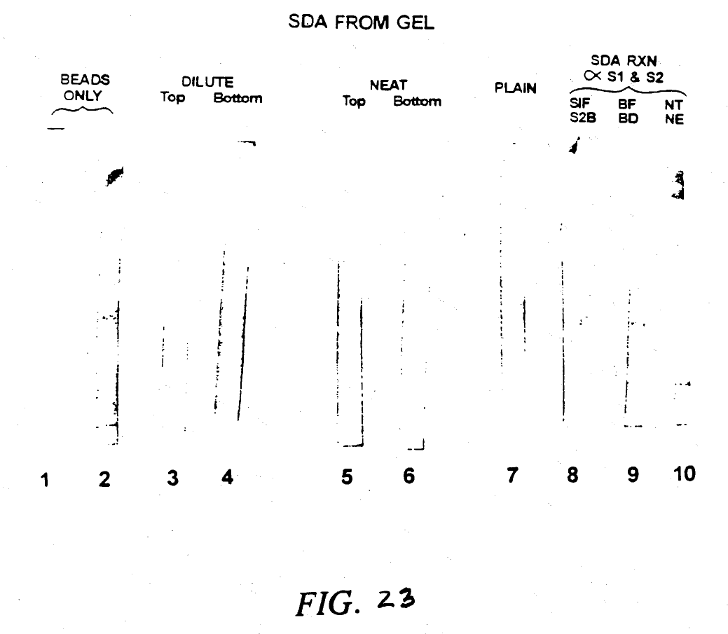

This set of experiments is conducted with composite extension primers that are labeled with biotin, fam or digoxigenin (FIGS. 21 and 22). Bumper primers are the same sequence as provided by Becton Dickinson and Company (Franklin Lakes, N.J). The sequences of the target, the bumper primer and the composite extension primer are as follows:

| | |
|---|---|
| Bumper primers: | |
| B1: 5′-CGATCGAGCAAGCCA | SEQ ID NO:5 |
| B2: 5′-CGAGCCGCTCGCTGA | SEQ ID NO:6 |
| Composite extension primers: | |
| S1: 5′-fam/dig-ACCGCATCGAATGCATGTCTCGGGTAAGGCGTACTCGACC | SEQ ID NO:7 |
| S2: 5′-biotin-CGATTCCGCTCCAGACTTCTCGGGTGTACTGAGATCCCCT | SEQ ID NO:8 |
| Target sequence: | |
| 5′-TGGACCCGCCAACAAGAAGGCGTACTCGACCTGAAAGACGTTATCCACCAT | SEQ ID NO:9 |
| ACGGATAGGGGATCTCAGTACACATCGATCCGGTTCAGCG | |

The reaction is set up per the thermophilic Strand Displacement Amplification (tSDA) protocol developed by Becton Dickinson and Co. The target organism is Mycobacterium tuberculosis. For pilot studies, an artificial target template comprising the 91nt sequence of the M. tuberculosis genome, defined by the Becton Dickinson outer (bumper) primers, is used. Amplification conditions used are identical to those used by Becton Dickinson for tSDA.

The membrane used for this procedure is nitrocellulose, purchased from Millipore Corporation, Bedford, Mass. A stripe of streptavidin at a concentration of 1 mg/mL is applied at a rate of 1 μL/cm via a linear reagent striper (IVEK Corporation, No. Springfield, Vt.) 1 cm from the bottom edge of the membrane. After application of the streptavidin, the membrane is allowed to dry and then blocked for non-specific binding by 0.5% casein in 100 mM Tris, pH 7.4. The membrane is washed twice with water (ddH₂O) and allowed to dry.

Next, 3 μL of anti-S1 extension primer (complementary to S1 without the biotin label) and/or S2 extension primer (complementary to S2 without the dig or fam label) is spotted onto a second membrane. The second membrane is then sandwiched onto the first membrane in order to capture free primers that compete with the product for the microparticles or streptavidin capture zone.

The coated microparticles are prepared as described above by incubating either anti-digoxigenin F(ab′)₂ or anti-fam monoclonal IgG with a suspension of microparticles. The coated microparticles are diluted 1:2 with a 35% sucrose solution, and 3 μL or the solution is applied directly to the membrane and dried.

The bifunctionally labeled reaction product (10 μL) is added to 45 μl SDA buffer, then applied (50 μL) to the previously striped membrane. Application of the sample requires the bifunctionally labeled product and the competing primers to pass through the anti-primer coated membrane and the dried microparticles. When the target is present, there is a visible line on the membrane. When the target is not present, there is absence of a visible fine. The results of one such experiment are shown in FIG. 23.

### EXAMPLE 3

### Inhibition Assay: Loss of Visible Signal on Lateral Flow Membrane

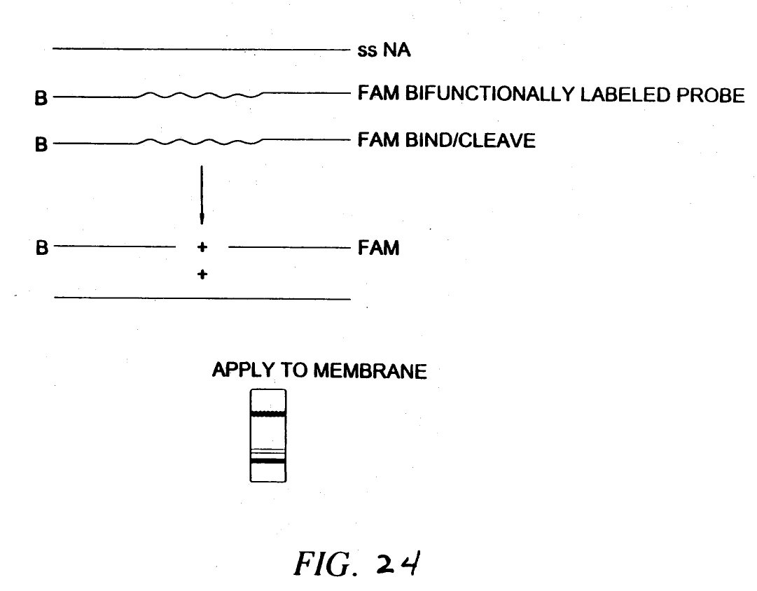

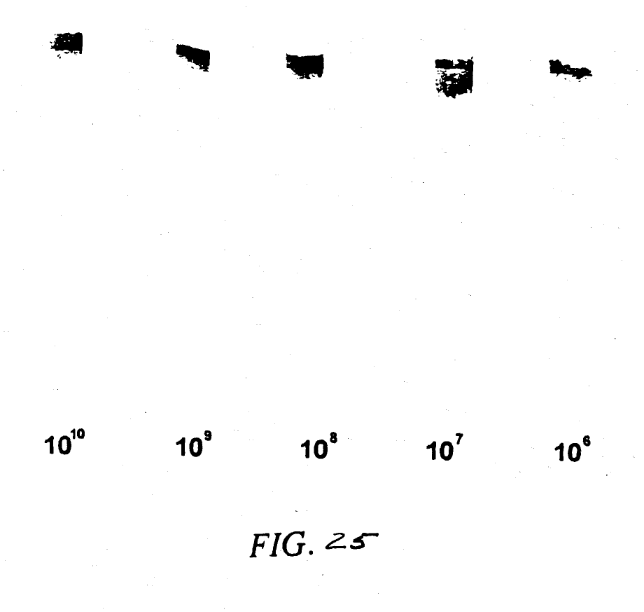

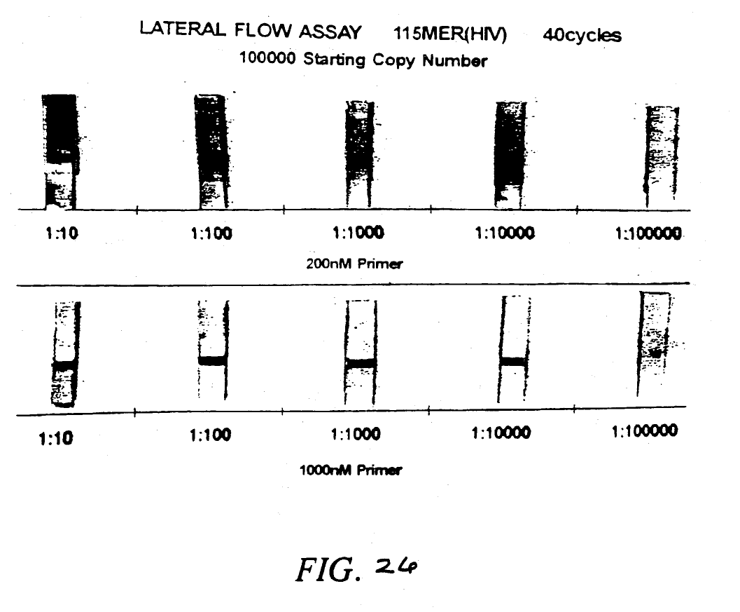

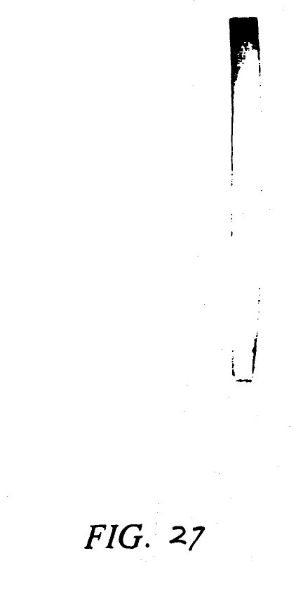

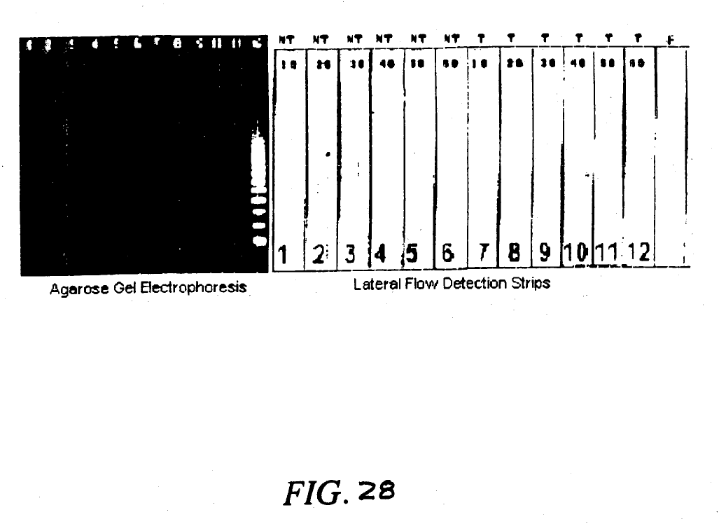

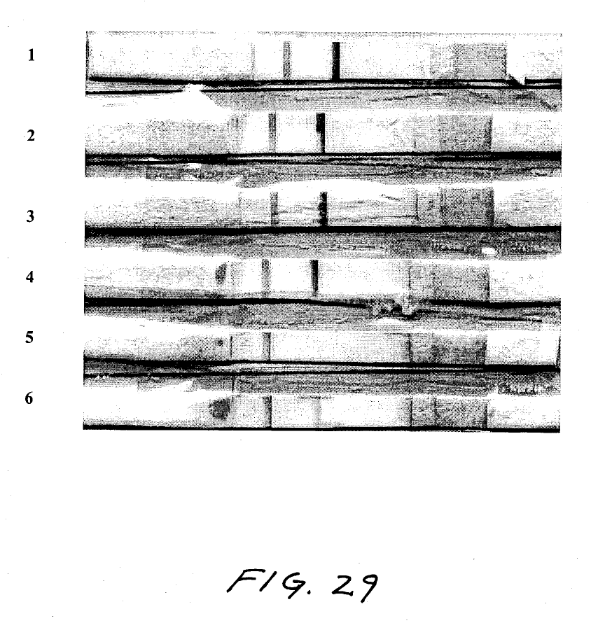

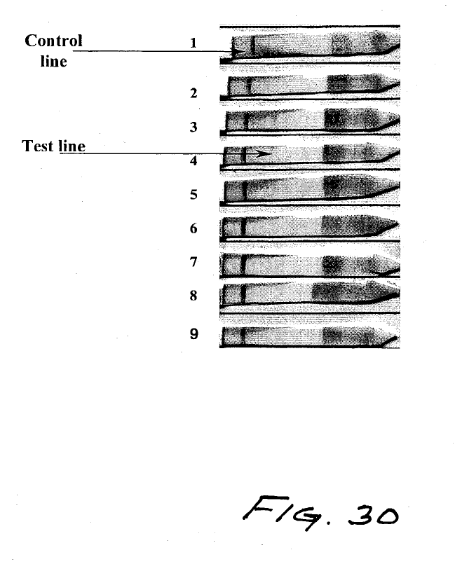

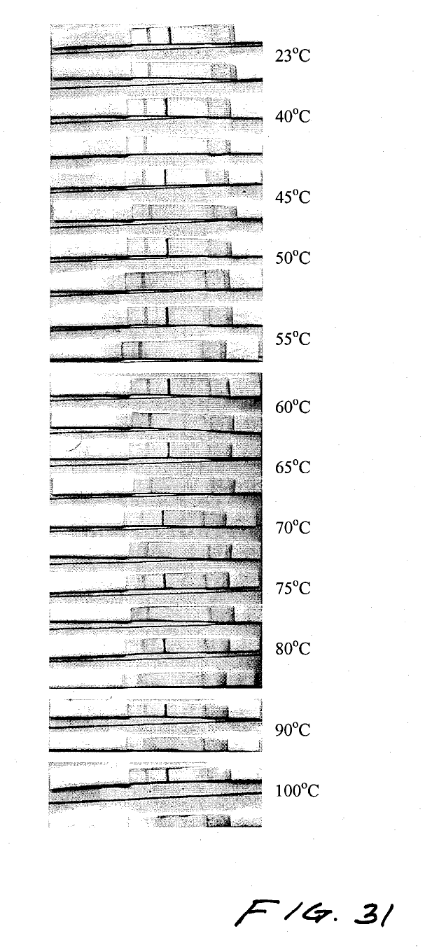

Cycling probe technology involves a nucleic acid probe that incorporates DNA-RNA-DNA sequences designed to hybridize with the target sequences. See, for example, FIG. 24. The probe is bifunctionally labeled with biotin and fam. If the probe hybridizes with the target generating double stranded nucleic acid, RNase H in the reaction buffer cleaves the probe. This cleavage results in loss of signal when applied to a membrane containing a capture zone of streptavidin and anti-fam coated colored microparticles. If the target is not present, there is a visible line on the membrane.

The specific probe and target employed in the instant example have been designed by ID Biomedical Corporation for use in detecting Mycobacterium tuberculosis. The probe (SEQ ID NO: 10) is a chimeric construct containing both DNA and RNA sequences with labels on the 5′ (fam) and the 3′ (biotin) ends of the DNA portion of the probe. The binding of the probe to a single strand of target generates double stranded nucleic acid which is cleaved with RNase H, thus eliminating the bifunctionality of the probe. The sequence of the probe is described below:

| | |
|---|---|
| FARK2S3B probe: | |
| 5′-fam-AAAGATGTagagGGTACAGA-biotin-3′ | SEQ ID NO:10 |
| (lower case indicates ribonucleoside | |
| bases) | |
| ARK2-T synthetic target: | |
| 5′-AATCTGTACCCTCTACATCTTTAA-3′ | SEQ ID NO:11 |

The reaction is completed following the protocol provided by ID Biomedical Corporation. The membrane used for this procedure is nitrocellulose, purchased from Millipore Corporation, Bedford, Mass. A stripe of streptavidin at a concentration of 1 mg/mL is applied at a rate of 1 μL/cm via a linear reagent striper (IVEK Corporation, No. Springfield, Vt.) 1 cm from the bottom edge of the membrane. After application of the streptavidin, the membrane is allowed to dry and then blocked for non-specific binding by 0.5% casein in 100 mM Tris, pH 7.4. The membrane is washed twice with water (ddH₂O) and allowed to dry. The microparticles used are anti-fam coated microparticles prepared as described above using anti-fam monoclonal IgG.

The reaction product (10 μL) is added to 5 μL of 0.1% anti-fam coated microparticles (0.1%) and 35 μL of water, then applied (50 μL) to the previously striped membrane. The binding of the bifunctionally labeled probe to the target, followed by cleavage of the probe by RNase H, results in loss of the bifunctionality of the probe. When the target is present, the absence of a visible line on the membrane exists. When the target is not present, the bifunctionally labeled probe is able to bind the anti-fam coated microparticles and the streptavidin bound to the membrane, resulting in a visible line. The results of one such experiment are shown in FIG. 25.

### EXAMPLE 4

### Detection of Bifunctionally Labeled Amplified Product

The membrane used for this procedure is nitrocellulose, purchased from Millipore Corporation, Bedford, Mass. A stripe of streptavidin at a concentration of 1 mg/ml is applied at a rate of 1 μL/cm via a linear reagent striper (IVEK Corporation, No. Springfield, Vt.) 1 cm from the bottom edge of the membrane. After application of the streptavidin, the membrane is allowed to dry and then blocked for non-specific binding by 0.5% casein in 100 mM Tris, pH 7.4. The membrane is washed twice with water (ddH₂O) and allowed to dry.

The amplification product is added to the membrane with colored receptor coated beads at dilutions of 0.001-1.0% microparticles/mL. This mixture is allowed to wick up the membrane. Positive reactions result in a colored line where the capture material is applied. Amplification reactions without the target sequence added to the reaction serve as negative controls. The results of this lateral flow assay are illustrated in FIG. 26.

If the target and control nucleic acid sequences are present, the receptor-bound microparticles interact with hapten(s) to capture the amplified nucleic acid. The result is a line of dyed particles visible on the membrane for the target and a line for the control nucleic acids. If the target is not present, the dyed particles for the target are not captured and are not visible. When the result of the analysis is negative, the control nucleic acid sequences must be visible indicating that the extraction and amplification were performed correctly.

### EXAMPLE 5

### Detection by Amplification with a Single Labeled Primer Followed by Hybridization with a Probe that Contains a Single Label

The target nucleic acid sequence is amplified by PCR using 200-1000 mM primer concentration, GeneAmp EZ rTth RNA PCR kit (Perkin Elmer Corp., Alameda, Calif.) and 10⁶ copies/mL of the target HIV RNA sequence. Forty PCR cycles, each cycle being 60° C. for 15 minutes, 95° C. for 15 seconds, and 55° C. for 60 seconds, are run.

The sequences of the primers are as follows:

| | |
|---|---|
| SK38 Dig Primer: | |
| 5′-dig-ATATCCACCTATCCCAGTAGGAGAAAT-3′ | SEQ ID NO:12 |
| SK39 Primer: | |
| 5′-TTTGGTCCTTGTCTTATGTCCAGAATGC-3′ | SEQ ID NO:13 |

Specific PCR reaction conditions are described below:

| Reagent | Final concentration | |
|---|---|---|
| 5X EZ Buffer | 1X | |
| Mn(OAc)₂ | 3 | mM |
| rTth polymerase | 5 | U |
| dntp's | 240 | μM each |
| SK38 | 1 | μM |
| SK39 | 1 | μM |

rTth DNA Polymerase (Perkin Elmer N808-0097)

The SK38 Dig - - - SK39 amplicon (5 μl) is incubated with 5 μL of 25 μM (125 pmol) SK39 biotin at 95° C. for 1 minute, and then at 55° C. for 1 minute. The amplicon binds to the anti-digoxigenin-coated microparticles and wicks through the membrane to the streptavidin line where it is captured by the interaction of biotin and streptavidin. The result is a visible line of colored microparticles.

In the negative control, the procedure is performed as described above, but without the addition of the target sequence. Without the presence of the target sequence in the amplification reaction, the bifunctionally labeled amplicon is not generated and the visible line of detection is not present. The results of one such experiment are shown in FIG. 27.

### EXAMPLE 6

### Extraction of Nucleic Acids with Guanidine Thiocyanate onto Glass (Silicon Dioxide) and Subsequent Amplification without Elution from Silicon Dioxide

A column was constructed using Ansys 0.4 mm membrane as a filter to contain the silicon dioxide and a syringe apparatus to pull buffer through the column in approximately 15 seconds. 50 μL serum, 2 μL SiO₂ (0.5 mg/μL), and 450 μL guanidine thiocyanate (GuSCN) lysis buffer are mixed by vortexing and then incubated at room temperature for 10 minutes. The specific lysis buffer for the instant set of experiments contains 14.71 g GuSCN (4M final), 0.61 mL “Triton X-100,” and 5.5 ml 0.2M EDTA (pH 8.0), and is q.s. to 31.11 mL with 0.1M Tris-HCl to pH 6.4. The silicon dioxide is washed twice with 500 μL 70% EtOH.

Next, the filter with SiO₂ is removed from the column and the SiO₂ is washed off of the membrane using 20 μL of water (ddH₂O). 5 μL of the silicon dioxide slurry is added to a PCR reaction using standard protocol for HIV model system, as detailed supra in Example 5.

### EXAMPLE 7

### Cascade Rolling Circle Amplification

The use of cascade rolling circle amplification (CRCA) and labeled primers for detection of target nucleic acid sequences was established in collaboration with Dr. David Thomas (Oncormed, Inc.). Amplicon from an HIV DNA plasmid model system was bifunctionally labeled during CRCA using tagged primers and subsequently detected by lateral flow chromatography (see FIG. 28). The target sequence was amplified 6 individual times at 10 minute increments. That is, amplification was performed for 10, 20, 30, 40, 50 and 60 minutes, respectively. FIG. 28 shows that the results of agarose gel electrophoresis show no visible results except for the target that was amplified for 60 minutes. Lateral flow chromatography detection strips demonstrate visual detection after 40 minutes of target amplification and a strong visual signal for both the 50 and 60 minute amplifications. These results support the use of an isothermal amplification platform with the self-contained device disclosed herein.

### EXAMPLE 8

### Preparation of Ligand-Bound Microparticles

(A) In one embodiment, the microparticles were anti-digoxigenin F(ab′)₂-coated microparticles. To prepare the anti-digoxigenin-coated microparticles, 0.25 to 1.0 mg/mL of anti-digoxigenin F(ab′)₂ was incubated with a suspension containing a final concentration of 1.0% microparticles/mL. The microparticles and digoxigenin F(ab′)₂ were allowed to react for 15 minutes prior to treatment with an activating agent such as EDAC (1-ethyl-3-(3-dimethylaminopropyl) carbodiimide) for covalent binding. The microparticles were treated with EDAC at a final concentration of 0.0 to 2.5 mM. The F(ab′)₂ and microparticles were mixed and incubated at room temperature for one hour. Unbound F(ab′)₂ was removed by successive washes and the coated microparticles are resuspended in storage buffer.

(B) In another embodiment, proteins (IgG or β-galactosidase) were conjugated to 300 nm carboxylate-modified microspheres (Seradyn) by the standard covalent coupling method using the standard 1-ethyl-3-(3-dimethylaminopropyl) carbodiimide hydrochloride (Pierce). The final conjugates were suspended in 25 mM TRIS (pH 8.4) 100 mM NaCl, and 1% Fish Skin Gelatin (FSG) containing 0.1% NaN₃. The microparticles were added to the labeling zone matrix, which was borosilicate glass fiber filters (AccuFlow™ P or G; Schleicher & Schuell), cellulose filters (P/N S70011, Pall Gelman Sciences, Ann Arbor, Mich.), or similar materials.

### EXAMPLE 9

### Design of Detection Probes

Detection probe oligonucleotide sequences were designed and selected by the standard protocol using Oligo 5 (Molecular Biology Insights, Inc., Cascade, Colo.). The probes were incorporated into a porous medium such as glass (e.g., borosilicate glass fiber), cotton, cellulose, polyester, rayon, polyethersulfone, polyethylene or other suitable medium. Detection probe mixes were diluted in the appropriate buffer (e.g., 25 mM TRIS, pH 8.4, 100 mM NaCl, 1% Fish Skin Gelatin containing 0.1% NaN₃) and added to the porous membrane. The medium was allowed to dry for at least 0.5 hr. at 30° C. in a forced air oven prior to cutting and assembly with the other lateral flow test strip components.

### EXAMPLE 10

### Preparation of Lateral Flow Test Strips

Preparation of Sample Receiving Zone: In this example, the sample receiving zone contains first and second oligonucleotide probes coupled to first and second binding partners, respectively. To prepare the sample receiving zone, the first and second probes are mixed together, diluted in an appropriate buffer to a final concentration of 1.25 μM, and then added to the sample receiving zone membrane. The membrane was allowed to dry for at least 0.5 hours at 300° C. in a forced air oven prior to assembly with the other lateral flow test strip components.

Preparation of Labeling Zone: Proteins (IgG or β-galactosidase) were conjugated to 300 nm carboxylate-modified microspheres (Seradyn) by the standard covalent coupling method using the standard 1-ethyl-3-(3-dimethylaminopropyl) carbodiimide hydrochloride (Pierce). The final conjugates were suspended in 25 mM TRIS, pH 8.4, 100 mM NaCl, 1% Fish Skin Gelatin (FSG), containing 0.1% NaN3. The conjugate release pad consisted of borosilicate glass fiber filters (AccuFlow™ P or G; Schleicher & Schuell) or cellulose filters (P/N S70011, Pall Gelman Sciences, Ann Arbor, Mich.) or similar materials.

Preparation of Capture Zone Membrane: The nitrocellulose was a large pore direct cast nitrocellulose with a polyester backing. This medium has a caliper of 270 μm and a capillary rise of 75-180 sec/4 cm deionized H₂O. The nitrocellulose used was the Hi-Flow™ membrane from Millipore. Anti-Fluorescein isothiocyanate (anti-FITC), [F(ab′)₂ fragments (Dako) and anti-β-galactosidase IgG (Cappel) were separately and exhaustively dialyzed against 10 mM phosphate buffered saline, pH 7.3 with a Slide-A-Lyzer® (Pierce). These antibodies were striped at a concentration of 1.0 mg/mL on the nitrocellulose using the linear reagent dispensing system (Striper/Digispense 2000 System (IVEK Corporation, North Springfield, Vt.). The Striper Controller was typically set for a rate of 40 mm/sec. The Digispense 2000 Controller was set at a dispense rate of 4.0 μL/sec. Membranes were generally blocked with 0.1% Casein in Tris buffered saline (pH 7.3) for 30 minutes, followed by a rinse with 0.05% Tween 20 and a final rinse in distilled water. Final drying was in a forced air oven at 30° C. for 30 minutes. The strips were stored at 23° C.±3° C. in a desiccated chamber until ready for use.

Assembly of the lateral flow test strip: An acrylic pressure sensitive adhesive, supported with 74 lb. white polypropylene coated silicone release liner (0.01 inch, GL-187; G & L Precision Die Cutting, San Jose, Calif.), was used as a backing. A strip of the capture zone membrane containing the test and control capture moieties is affixed to the adhesive side of the laminate. In this example, the capture zone membrane extends approximately the length of the laminate. The sample receiving zone is affixed to the proximal end of the capture zone membrane, and an absorbent pad is affixed to the distal end of the capture zone membrane. The absorbent pad can be cotton linter paper (#470; Schleicher & Schuell), bonded cellulose acetate (Transpad™ wicks R-18552, Filtrona Richmond Inc., Richmond, Va.) or cellulose absorbent (Ahlstrom, Mt. Holly Springs, Pa.).

The test strips were generally cut into 5 mm strips with the Matrix 2360 Programmable Shear (Kinematic Automation, Twain Harte, Calif.). In some cases the strips were cut by hand. For some studies, the lateral flow laminates were enclosed in ARcare® 7759 (Adhesives Research, Inc. Glen Rock, Pa.), which is a 1 mil clear polyester film carrier containing AS-110 Acrylic, a medical pressure-sensitive adhesive-coated on one side of a film for bonding and also containing a 2 mil siliconized clear polyester release liner.

### EXAMPLE 11

### Detection of E. Coli Amplicon

Preparation of Lateral Flow Strips: Lateral flow laminates were cut into 3 mm strips by hand or with the Matrix 2360 Programmable Shear (Kinematic Automation, Twain Harte, Calif.). In addition the laminates were enclosed in a 1 mil clear polyester film carrier containing AS-110 Acrylic, a medical pressure-sensitive adhesive-coated on one side of a film for bonding and also containing a 2 mil siliconized clear polyester release liner (ARcare® 7759; (Adhesives Research, Inc., Glen Rock, Pa.).

Amplification: In a typical experiment an E. coli lacZ gene was amplified using a NASBA procedure. The amplification primers designed were designated primer #5085 (primer with T7-promoter sequence underlined) having the sequence 5′-AATTCTAATACGACTCACTATAGGGAGAGGACGGATAAACGGAACTGGA (SEQ ID NO. 14) and primer #5086 having the sequence 5′-ATGATGAAAACGGCAACC (SEQ ID NO. 15). The two detection probes used in this assay were designated #5087 and #5088 and represented by 5′-FITC-GGTCGGCTTACGGCGGTG-phosphate (SEQ ID NO. 16) and 5′-CTGTATGAACGGTCTGGTCTTTG-Biotin (SEQ ID NO. 17), respectively.

The NASBA reaction mix contained 200 nM each amplification primer and 70 mM KCl. Master and enzyme mixes are prepared using the Nuclisens Basic Kit (Organon Teknika, Boxtel, NL).

TABLE 1
NASBA Reagent Concentrations

| Reagent | E. coli Stock Conc. | | E. coli Final Conc. | |
|---|---|---|---|---|
| Tris-HCl, pH 8.5 | 2000 | mM | 80 | mM |
| KCl | 2000 | mM | 50 | mM |
| MgCl₂ | 1000 | mM | 12 | mM |
| DTT | 500 | mM | 10 | mM |
| dNTP mix | 25 | mM (each) | 1 | mM |
| rNTP mix | 25 | mM (each) | 2 | mM |
| Primer mix | 25000 | nM (each) | 200 | nM |

| | | |
|---|---|---|
| Sorbitol | 75% | 15% |
| DMSO | 100% | 15% |
| BSA | In enzyme mix | |

TABLE 2
Enzyme Mix Concentrations

| | E. coli | | E. coli | |
|---|---|---|---|---|
| Reagent | Stock Concentration | | Final Concentration | |
| RNA Guard | 26 | U/μL | 0.25 | U/μL |
| BSA | 10000 | μg/μL | 100 | μg/μL |
| AMV RT | 22.98 | U/μL | 8.0 | U/μL |
| T7 RNA Pol. | 61 | U/μL | 40 | U/μL |
| RNase H | 1 | U/μL | 0.2 | U/μL |

Amplification was performed as described by the manufacturer with a heat step at 65° C. for 2 minutes followed by cooling to 40° C. for 15 seconds. Enzyme was added and the reaction allowed to proceed for 90 minutes at 40° C.

Detection: In order to determine the reactivity of the complete lateral flow assay system of this invention, lateral flow strip laminates were assembled comprising porous media, antibody-striped nitrocellulose, NeutrAvidin-coated microparticles and β-galactosidase-coated microparticles and oligonucleotide probes. The amplified target nucleic acid was diluted in 50 mM Tris-HCl, pH 8.0, 8.0 mM MgCl₂, 0.025% Triton X-100 and heated to 90° C. prior to applying to the lateral flow device. Detection primers #5087 and 5088 were each used at a concentration of 1.25 μM. In this experiment, various time intervals for heating of the amplified product were investigated. The time intervals prior to the addition of hot amplified product were 0, 3 and 10 minutes. A negative control was subjected to heating for 5 minutes. In addition, a non-sealed positive control assay device was used.

Results: This example illustrates the performance of sealed lateral flow test strips. The results of this assay are shown in FIG. 29. In FIG. 29, strips 2-6 were laminated with a clear polyester having an acrylic adhesive; strip 1 was the positive control strip (i.e., no laminate coating); strips 2-4 were subjected to a temperature of 90° C. at 0, 3 and 10, minutes respectively; strip 5 was a laminated negative control after 5 minutes at 90° C.; and strip 6 shows the lateral flow limit-of-detection of a sample subjected to 90° C. for 5 minutes. This Example demonstrates that lateral flow test strips of this invention, constructed in a ready-to-use manner as described, can be completely enclosed to prevent amplicon contamination without loss of strip integrity.

### EXAMPLE 12

### Direct Detection of Salmonella by Hybridization

In this example, Salmonella invA target was detected following nucleic acid extraction and hybridization with complementary labeled probes. This hybridization test exploits the ability of complementary nucleic acid sequences to specifically align and associate to form stable double-stranded complexes.

Growth and treatment of Salmonella typhimurium: One colony of S. typhimurium (X3-002) was picked from plated culture into 3 ml of Trypticase Soy Broth (TSB). This was incubated overnight in a 37° C. shaking water bath to stationary phase. A 1/100 dilution of the overnight culture was made into 100 ml of fresh TSB. The new culture was placed in the water bath and grown for approximately 4 hours to late log phase. The final concentration at the time of testing was approximately 10⁸ CFU/ml. Generally, S. typhimurium was centrifuged at 8,000×g for 5 minutes at 25° C.±3° C. in order to pellet the cells. A pellet ranging from 200 to 400 μL was obtained.

Type VII Subtilisin from Bacillus lichenformis (10.6 units/mg solid; Sigma P5380; Lot 12K1719) was diluted to 10 mg/ml in Sigma purified water and used in a v/v bacterial pellet to enzyme ratio of 6:1. Amplification grade DNase I; EC 3.1.21.1 (Sigma AMP-D1; lot 082K9301) containing 1,000 units of DNase was used in some cases to treat the sample. It was important in this instance to treat with DNase prior to using the alkaline protease if the two were to be used in the same treatment protocol. Series II Lysis Buffer Stock Buffer (Xtrana, Inc.) containing LiCl facilitated the lysis of the bacterial pellet and was used in a pellet to buffer ratio of 1:2. Treatment was conducted at 250° C.±3° C. Sonication was performed in some cases from 1 to 3 minutes at 25° C.±3° C.

Detection: Lateral flow was performed according to the standard protocol with 3 mm wide strips impregnated with a mixture of blue NeutrAvidin™ and red β-galactosidase-conjugated microparticles. The typical volume used for the strip was 40 μL. In most cases, it was necessary to “chase” the suspension with an additional 20 μL of 50 mM Tris-HCl, pH 8.0, 8.0 mM MgCl₂ and 0.25% Triton X-100 (Lateral Flow Buffer).

Results: FIG. 30 shows the results obtained following treatment of S. typhimurium with Series II lysis buffer, sonication and proteolysis. The results are represented in increasing levels of detection probe mix concentration. This example demonstrates that nucleic acid testing can benefit from direct detection of nucleic acids from pathogenic microorganisms following nucleic acid extraction. This approach generally shortens the time-to-results, enabling early decision making and reducing costs. The application of nucleic acid hybridization following the lysis of a pathogenic microorganism and subsequent detection by lateral flow has not been previously reported.

### EXAMPLE 13

### Lateral Flow Detection of Nucleic Acid Targets at High Temperatures

This example demonstrates that the lateral flow devices of this invention can be used to conduct lateral flow tests for nucleic acids at elevated temperatures.

In a typical experiment, complete lateral flow strips were placed at various temperatures in a forced air oven for at least two minutes to allow for equilibration. The targets of interest Were added and the reactions allowed to proceed at the respective temperatures. In each case the negative control used was represented by the lateral flow buffer containing the detection probe mixture.

The oligonucleotide tested was a 50-mer having the sequence (SEQ ID NO. 18):

5′-FITC-ATCTTAGTCGGAAATCGTATTCAAGTTTATATGACCAGGCAGTAGATACT-Biotin.

The sequence was stabilized with a complementary oligonucleotide of the same length. Effect of heat on the integrity of complete lateral flow detection systems in DNA testing is shown in FIG. 31. In FIG. 31, one positive strip (+) and one negative strip (−) is shown for each temperature tested.

The results indicate that this lateral flow assembly for the detection of nucleic acid targets is fully functional at temperatures ranging from 20° C.±3° C. to 90° C.±3° C. Those skilled in nucleic acid analysis can use this treatment to perform stringency experiments for approximating Watson-Crick complementarity. This demonstrates the first use of lateral flow for this purpose.

The instant invention provides a rapid, simple and accurate method of detecting amplified target nucleic acid sequences with a self-contained device. Sensitivity and specificity of the assay are based on labeling of the target, by incorporating a label or by subsequent hybridization of a labeled probe during the amplification process. The method does not require costly and sophisticated equipment or specially trained personnel, nor does it pose any health hazard.

While the above description contains many specificities, these should not be construed as limitations on the scope of the invention, but rather an exemplification of the preferred embodiment thereof. Many other variations are possible, such as amplifying several target samples in the same reaction mixture, isothermal amplification, utilizing newly discovered polymerases and ligases, etc. Thus the scope of the invention should be determined by the appended claims and their legal equivalents, rather than by the example given.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

We claim:

1. A lateral flow assay device for detecting the presence or absence of at least one single-stranded target nucleic acid in a fluid sample, said device having a first and second end and comprising:
a sample receiving zone at or near said first end for receiving an aliquot of said sample and comprising a porous material having first and second oligonucleotide probes coupled to first and second binding partners, respectively, wherein said probes specifically hybridize to said target nucleic acid to form a complex having said first and second binding partners, said sample receiving zone being in lateral flow contact with
a labeling zone comprising a porous material having at least a first visible moiety reversibly bound thereto and coupled to a first ligand which specifically binds to said first binding partner to form a visible complex, said labeling zone being in lateral flow contact with
a capture zone comprising a microporous membrane which contains in a portion thereof a first capture moiety immobilized thereto which specifically binds said second binding partner, said capture zone being in lateral flow contact with
an absorbent zone positioned at or near the second end of said device,
wherein said visible complex is captured by said capture moiety in said portion of the capture zone.

2. The device of claim 1, wherein said sample receiving zone porous material retains said probes prior to contact with said fluid sample and releases said probes after contact with said fluid sample.

3. The device of claim 2, wherein said sample receiving zone porous material is selected from the group consisting of glass, cotton, cellulose, polyester, rayon, nylon, polyethersulfone, and polyethylene.

4. The device of claim 1, wherein said first and second binding partners are selected from the group consisting of antibodies or fragments thereof, proteins, haptens, antigens or fragments thereof, avidin, streptavidin, biotin, fluorescein isothiocyanate, folic acid, folate binding protein, protein A, protein G, immunoglobulins, digoxigenin, anti-digoxigenin F(ab′)₂, complementary nucleic acid segments, protein A, protein G, immunoglobulins, lectin, carbohydrate, enzymes, viruses, maleimides, haloacetyl derivatives, isotriocyanates, succinimidyl esters, sulfonyl halides, steroids, halogens and 2,4-dinitrophenyl.

5. The device of claim 1, wherein said labeling zone porous material is selected from the group consisting of glass, cotton, cellulose, polyester, polyethylene, rayon or nylon.

6. The device of claim 1, wherein said first visible moiety comprises a ligand coupled to a colored microparticle.

7. The device of claim 6, wherein said microparticle is selected from the group consisting of polymers or copolymers of olefinically unsaturated monomers, glass, acrylamide, methacrylate, nylon, acrylonitrile, polybutadiene, metals, metal oxides and their derivatives, dextran, cellulose, liposomes, red blood cells, pollens, and bacteria.

8. The device of claim 1, wherein said capture zone membrane comprises a microporous material selected from the group consisting of nitrocellulose, polyethersulfone, polyvinylidine fluoride, nylon, charge-modified nylon, and polytetrafluoroethylene.

9. The device of claim 1, wherein said first capture moiety is selected from the group consisting of antibodies or fragments thereof, proteins, haptens, antigens or fragments thereof, avidin, streptavidin, biotin, fluorescein isothiocyanate, folic acid, folate binding protein, protein A, protein G, immunoglobulins, digoxigenin, anti-digoxigenin F(ab′)₂, complementary nucleic acid segments, protein A, protein G, immunoglobulins, lectin, carbohydrate, enzymes, viruses, maleimides, haloacetyl derivatives, isotriocyanates, succinimidyl esters, sulfonyl halides, steroids, halogens and 2,4-dinitrophenyl.

10. The device of claim 1, wherein said capture zone is prepared by applying a solution containing said capture moiety to said membrane under conditions wherein the capture moiety becomes immobilized on said membrane, followed by drying said membrane.

11. The device of claim 10, wherein said solution is applied to said membrane in the form of a line.

12. The device of claim 1, wherein said labeling zone further comprises a second visible moiety reversibly affixed to said matrix and coupled to a second ligand, and said capture zone further comprises in a portion thereof a second capture moiety immobilized thereon which specifically binds said second ligand.

13. The device of claim 12, wherein said portion of said capture zone containing said first capture moiety is separate from said portion containing second capture moiety.

14. The device of claim 1, wherein said absorbent zone comprises a material selected from the group consisting of nitrocellulose, cellulose esters, glass, polyethersulfone, and cotton.

15. The device of claim 1, wherein said entire test strip except for a portion of said sample receiving zone is completely sheathed in a transparent film.

16. The device of claim 15, wherein said film is a polyester, polycarbonate, polystyrene, polypropylene, glycol modified polyethylene terphthalate, a heat resistant acrylic, or a butyrate.

17. The device of claim 1, wherein said sample receiving zone microporous material is affixed to a first end of the top side of said capture zone membrane, said labeling zone is affixed to the top side of said capture zone membrane and position between said sample receiving zone and said capture zone, and said absorbent pad is affixed to the top side of said capture zone membrane near the second end of said membrane.

18. The device of claim 17, wherein said capture zone membrane is affixed to the top side of a rigid or semi-rigid support.

19. The device of claim 18, wherein said rigid or semi-rigid support comprises polypropylene, poly(vinyl chloride), propylene, or polystyrene).

20. The device of claim 18, further comprising a heating sheet affixed to the bottom side of said rigid or semi-rigid support.

21. The device of claim 1, wherein said sample receiving zone, said labeling zone, said capture zone, and said absorbent zone are affixed to the top side of a rigid or semi-rigid support.

22. The device of claim 21, wherein said support comprises polypropylene, poly(vinyl chloride), propylene, or polystyrene).

23. The device of claim 21, further comprising a heating sheet affixed to the bottom side of said support.

24. The device of claim 1, further comprising a piercing means at said first end.

25. The device of claim 1 for detecting the presence of two or more target nucleic acids, wherein the sample receiving zone comprises a first and second oligonucleotide probe specific for each of said target nucleic acid, the labeling zone comprises a first visible moiety specific for each of said target nucleic acids and distinguishable from the other visible moieties, and the capture zone comprises a capture moiety specific for each of said target nucleic acids.

26. A lateral flow assay device for detection of the presence or absence of at least one target nucleic acid in a fluid sample, wherein said target nucleic acid is coupled to a first binding partner, said device comprising a test strip having a first and second end and comprising:
a sample receiving zone at or near said first end for receiving an aliquot of said sample and comprising a porous material having an oligonucleotide probe coupled to a second binding partner, wherein said probe is reversibly bound to said microporous material and specifically hybridizes to said target nucleic acid to form a complex comprising said first and second binding partners, said sample receiving zone being in lateral flow contact with
a labeling zone comprising a porous material having at least a first visible moiety reversibly bound thereto and coupled to a first ligand which specifically binds to said first binding partner to form a Visible complex, said labeling zone being in lateral flow contact with
a capture zone comprising a microporous membrane which contains in a portion thereof at least a first capture moiety immobilized thereto which specifically binds said second binding partner, said capture zone being in lateral flow contact with
an absorbent zone positioned at or near the second end of said test strip, wherein said visible complex is captured by said capture moiety in said portion of the capture zone.

27. The lateral flow assay device of claim 26 for detecting the presence of two or more target nucleic acids, wherein the sample receiving zone comprises an oligonucleotide probe specific for each of said target nucleic acids, the labeling zone comprises a first visible moiety specific for each of said target nucleic acids and distinguishable from the other visible moieties, and the capture zone comprises a capture moiety specific for each of said target nucleic acids.

28. A lateral flow assay device for detection of the presence or absence of at least one target nucleic acid in a fluid sample, wherein said target nucleic acid is coupled to a first and second binding partner, said device comprising a test strip having a first and second end and comprising:
a sample receiving zone at or near said first end of said test strip for receiving an aliquot of said sample and comprising a porous material, said sample receiving zone being in lateral flow contact with
a labeling zone comprising a porous material having at least a first visible moiety coupled to a first ligand which specifically binds to said first binding partner to form a visible complex, said labeling zone being in lateral flow contact with
a capture zone comprising a membrane which contains in at least a portion thereof at least a first capture moiety immobilized thereon which specifically binds said second binding partner, said capture zone being in lateral flow contact with
an absorbent zone at or near said second end of said test strip,
wherein said visible complex is captured by said capture moiety in said portion of the capture zone.

29. The lateral flow assay device of claim 28 for detecting the presence of two or more target nucleic acids, wherein the labeling zone comprises a first visible moiety specific for each of said target nucleic acids and distinguishable from the other visible moieties, and the capture zone comprises a capture moiety specific for each of said target nucleic acids.

30. A method for detecting the presence or absence of at least one target nucleic acid in a fluid sample, said method comprising:
(a) applying said sample to a sample receiving zone of a lateral flow test strip of a lateral flow assay device, wherein prior to said application said nucleic acid present in a double-stranded form are rendered single-stranded, wherein said sample wicks sequentially from said sample receiving zone to a labeling zone and to a capture zone of said test strip, said sample receiving zone comprising first and second oligonucleotide probes coupled to first and second binding partners, respectively, and reversibly bound to said test strip, wherein said probes are released from said test strip and specifically hybridize to said target nucleic acid upon contact with said sample to form a complex comprising said first and second binding partners,
said labeling zone comprising at least a first visible moiety coupled to a first ligand and reversibly bound to said test strip, wherein said first ligand specifically binds said first binding partner, and
said capture zone comprising a capture moiety immobilized on a portion of said test strip, wherein said capture moiety specifically binds said second binding partner; and
(b) detecting the presence of said first visible moiety in said portion of said capture zone.

31. The method of claim 30, wherein prior to step (a) said target nucleic acid is amplified.

32. The method of claim 31, wherein said amplification methodology is polymerase chain reaction (PCR), ligase chain reaction, Qβ replicase, strand displacement amplification (SDA), nucleic acid sequence-based amplification (NASBA), loop amplification (LAMP), ramification amplification (RAM), or cascade rolling circle amplification (CRCA).

33. The method of claim 27, wherein said assay device is affixed to a heating sheet, said method further comprising heating said device while said sample is wicking along said test strip.

34. The method of claim 27, wherein said device is heated to a temperature between about 20 and 95° C.

35. The method of claim 30, wherein said assay is performed under high stringency conditions.

36. The method of claim 30, wherein said assay is performed under low stringency conditions.

37. The method of claim 30, wherein said labeling zone further comprises a second visible moiety reversibly bound to said test strip and coupled to a second ligand, and said capture zone further comprises a second capture moiety immobilized on said test strip, wherein said second capture moiety specifically binds said second ligand.

38. The method of claim 30, wherein said entire test strip except for a portion of said sample receiving zone is sheathed in a transparent film.

39. The method of claim 30 for detecting the presence of two or more target nucleic acids, wherein the sample receiving zone comprises a first and second oligonucleotide probe specific for each of said target nucleic acid, the labeling zone comprises a first visible moiety specific for each of said target nucleic acids and distinguishable from the other visible moieties, and the capture zone comprises a capture moiety specific for each of said target nucleic acids.

40. A method for detecting the presence or absence of at least one target nucleic acid in a fluid sample, said method comprising:
(a) coupling said target nucleic acid to a first binding partner to provide a labeled target nucleic acid;
(b) applying said labeled target nucleic acid to a sample receiving zone of a lateral flow test strip of a lateral flow assay device, wherein prior to said application said nucleic acid present in a double-stranded form are rendered single-stranded, wherein said labeled target nucleic acid wicks sequentially from said sample receiving zone to a labeling zone and to a capture zone of said test strip, said sample receiving zone comprising an oligonucleotide probe coupled to a second binding partner and reversibly bound to said test strip, wherein said probes are released from said test strip and specifically hybridize to said labeled target nucleic acid upon contact with said labeled target nucleic acid to form a complex comprising said first and second binding partners,
said labeling zone comprising at least a first visible moiety coupled to a first ligand and reversibly bound to said test strip, wherein said first ligand specifically binds said first binding partner, and
said capture zone comprising a capture moiety immobilized on a portion of said test strip, wherein said capture moiety specifically binds said second binding partner; and
(c) detecting the presence of said first visible moiety in said portion of said capture zone.

41. The method of claim 40, wherein said target nucleic acid is coupled to said first binding partner by amplifying said target nucleic acid with a primer comprising said first binding partner.

42. The method of claim 40 for detecting the presence of two or more target nucleic acids, wherein the sample receiving zone comprises an oligonucleotide probe specific for each of said target nucleic acids, the labeling zone comprises a first visible moiety specific for each of said target nucleic acids and distinguishable from the other visible moieties, and the capture zone comprises a capture moiety specific for each of said target nucleic acids.

43. A method for detecting the presence or absence of at least one target nucleic acid in a fluid sample, said method comprising the steps of:
(a) coupling said target nucleic acid to a first and second binding partner to provide a labeled target nucleic acid;
(b) applying said labeled target nucleic acid to a sample receiving zone of a lateral flow test strip of a lateral flow assay device, wherein said labeled target nucleic acid laterally wicks sequentially from said sample receiving zone through a labeling zone to a capture zone of said test strip,
said labeling zone comprising at least a first visible moiety reversibly bound to said test strip and coupled to a first ligand, wherein said first ligand specifically binds said first binding partner, and
said capture zone comprising a capture moiety immobilized on a portion of said test strip, wherein said capture moiety specifically binds said second binding partner; and
(c) detecting the presence of said first visible moiety in said portion of said capture zone.

44. The method of claim 43, wherein said target nucleic acid is coupled to said first and second binding partners by amplifying said target nucleic acid with first and second primers comprising said first and second binding partners, respectively, wherein said amplified nucleic acid is rendered single-stranded prior to step (b).

45. The method of claim 43 for detecting the presence of two or more target nucleic acids, wherein the labeling zone comprises a first visible moiety specific for each of said target nucleic acids and distinguishable from the other visible moieties, and the capture zone comprises a capture moiety specific for each of said target nucleic acids.
